(19) United States
(12) Patent Application Publication
(10) Pub. No.: US 2023/0110899 A1
(43) Pub. Date: Apr. 13, 2023

(54) NOVEL COMPOSITIONS FOR TREATMENT OF CORONAVIRUS DISEASE

(71) Applicant: Laurent Pharmaceuticals Inc., Montreal (CA)

(72) Inventors: Radu Pislariu, Candiac (CA); Irenej Kianicka, Rosemère (CA); Danuta Radzioch, Repentigny (CA); Larry Lands, Hampstead (CA)

(21) Appl. No.: 17/913,118

(22) PCT Filed: Mar. 26, 2021
(86) PCT No.: PCT/CA2021/050401

§ 371 Date: Sep. 20, 2022

Related U.S. Application Data
(60) Provisional application No. 63/000,168, filed on Mar. 26, 2020.

(51) Int. Cl. A61K 31/167; A61K 47/20; A61K 9/00; A61K 9/08; A61K 9/10; A61K 9/14

(52) U.S. Cl. CPC A61K 31/167; A61K 47/20; A61K 9/0078; A61K 9/08; A61K 9/10; A61K 9/145

(57) ABSTRACT

The present invention provides for use of fenretinide, fenretinide analog or pharmaceutically acceptable salts for the preparation of medicaments useful for the treatment of SARS-coronavirus, ARDS and SARS-coronavirus associated pneumonia and hypoxemia. In addition, prophylaxis of SARS-coronavirus, ARDS and SARS-coronavirus associated pneumonia is also contemplated.

### CROSS-REFERENCE TO RELATED APPLICATIONS

The present application claims priority from U.S. Provisional Patent Application no. 63/000,168 filed Mar. 26, 2020, such application is expressly incorporated by reference herein for all purposes.

### FIELD OF THE INVENTION

The present invention relates to compositions and methods of use of fenretinide (4-hydroxyphenyl retinamide) and its associated analogs for the prophylaxis and/or treatment of coronavirus infection and its associated consequences.

### BACKGROUND OF THE INVENTION

In the past two decades, coronaviruses have caused two epidemic diseases, namely, severe acute respiratory syndrome coronavirus (SARS-coronavirus) and Middle East respiratory syndrome coronavirus (MERS-coronavirus). In December 2019, a new global outbreak emerged into a pandemic caused by a new SARS coronavirus (COVID-19 or SARS-CoV-2). Though appreciable efforts have been made in the past to identify treatments for SARS-coronavirus and MERS-coronavirus infections, there is a need for additional therapeutic interventions for these diseases and any subsequent sequalae that may arise from the infection.

In December 2019, patients presenting with cough, fever, and dyspnea with acute pneumonia due to an unidentified microbial infection were reported in Wuhan, China. Virus genome sequencing of five patients with pneumonia revealed the presence of a previously unknown β coronavirus strain (β-CoV) showing identity to the sequence of bat-derived severe acute respiratory syndromes (SARS)-like coronaviruses, including MERS-coronavirus. Patients with COVID-19 show clinical manifestations that include fever, non-productive cough, dyspnea, myalgia, fatigue, normal or decreased leukocyte counts, and radiographic evidence of pneumonia, which are similar to the symptoms of SARS-coronavirus and MERS-coronavirus infections. (Li, X. et al., Journal of Pharmaceutical Analysis, https://doi.org/10.1016/j.jpha.2020.03.00, 2020). As reported by Huang et al, although most patients with COVID-19 are thought to have a favorable prognosis, older patients and those with chronic underlying conditions may have worse outcomes. Patients with severe illness may develop dyspnea and hypoxemia within one week after the onset of the disease, which may quickly progress to acute respiratory distress syndrome (ARDS) or end-organ failure. (Huang, C. et al, The Lancet, https://doi.org/10.1016/S0140-6736(20)30183-5, 2020).

Cytokine storm and viral evasion of cellular immune responses are thought to play important roles in disease severity. Indeed, one of the main mechanisms for ARDS is the cytokine storm, the uncontrolled systemic inflammatory response resulting from the release of large amounts of pro-inflammatory cytokines (IFN-α, IFN-γ, IL-1β, IL-6, IL-12, IL-18, IL-33, TNF-α, TGFβ, etc.) and chemokines (CCL2, CCL3, CCL5, CXCL8, CXCL9, CXCL10, etc), which may lead to lung injury and death (Li, X. et al., Journal of Pharmaceutical Analysis, https://doi.org/10.1016/j.jpha.2020.03.00, 2020).

Neutrophilia was also found in both the peripheral blood and lung of patients with SARS-CoV-2 coronavirus infection. The severity of lung damage correlated with extensive pulmonary infiltration of neutrophils and macrophages and higher numbers of these cells in the peripheral blood in patients with MERS-CoV. Patients with COVID-19 pneumonia who had developed ARDS had significantly higher neutrophil counts than did those without ARDS, suggestive of an overreactive immune response that could also contribute to the cytokine storm. Age was also a factor related to mortality, older patients being more frequently associated with ARDS, which could also be explained by a less efficient immune responses. (Wu, C. et al, JAMA Internal Medicine, https://doi.org/10.1001/jamainternmed.2020.0994).

Increased alveolar—capillary permeability to fluid, proteins, neutrophils and red blood cells, to oedema in the lung interstitium and the alveoli, is the hallmark of ARDS. When alveolar oedema develops, reabsorption of the oedematous fluid depends on junctions between epithelial alveolar cells and ion transport channels (sodium channel and/or Na+/K+-ATPase function), which are affected in viral infection, resulting in impaired alveolar fluid clearance in patients with ARDS. (Matthay, M. et al., Nature Reviews Disease Primers, https://doi.org/10.1038/s41572-019-0069-0).

Pulmonary infiltration of neutrophils, viral evasion, cytokine storms and alveolar oedema are all consequences of an overreactive immune-inflammatory response leading to pulmonary distress and need for mechanical ventilation in a large percentage of ARDS patients. The prophylaxis and/or treating of SARS-coronavirus infections is a major challenge for clinicians. No pharmacological therapies of proven efficacy yet exist. Corticosteroids were widely used during the outbreaks of SARS-CoV-2 coronavirus and then in MERS-coronavirus infections, without conclusive results. (Russell, C. D. et al., The Lancet, https://doi.org/10.1016/S0140-6736(20)30317-2, 2020; Huang, C. et al, The Lancet, https://doi.org/10.1016/S0140-6736(20)30183-5, 2020).

Therefore, COVID-19 is a rapidly emerging viral infection and limited therapeutic options currently exists for treatment. While most people (80%) recover, about 20% will experience severe disease that may lead to ARDS and potential need for mechanical ventilation, creating an unsustainable burden for the health care system and a rapidly escalating crisis. The main cause for ARDS is an overreactive inflammatory response (cytokine storm). Current anti-inflammatory treatments (e.g., corticosteroids) are immune-suppressive and do not appear to have a benefit in early stage of the disease where an active immune response is important to clear the virus. There is a need for therapies able to keep an effective host defense response against the virus, while keeping the inflammation from overreacting and progressing toward ARDS.

In view of the above there is a need for pharmaceutical compounds and composition for the prophylaxis and treating of SARS-coronavirus types of infections and their complications.

### SUMMARY OF THE INVENTION

In one aspect, the present invention provides for a method of treating a SARS-coronavirus infection in a human comprising administration to said human of a therapeutically effective amount of fenretinide, fenretinide analog or pharmaceutically acceptable salt thereof. In one embodiment, the therapeutically effective amount of fenretinide, fenretinide analog or pharmaceutically acceptable salt thereof comprises 1 mg to 1000 mg of fenretinide. In a further embodiment, the therapeutically effective amount of fenretinide, fenretinide analog or pharmaceutically acceptable salt thereof comprises 10 mg to 300 mg of fenretinide. In an alternative embodiment the therapeutically effective amount of fenretinide, fenretinide analog or pharmaceutically acceptable salt thereof gives rise to a plasma concentration in said human of 0.5 μM to about 10 μM of fenretinide. In a further embodiment the therapeutically effective amount of fenretinide, fenretinide analog or pharmaceutically acceptable salt thereof gives rise to a plasma concentration in said human of 1 μM to about 3 μM of fenretinide.

In another aspect, the present invention provides for a method of treating a SARS-coronavirus infection in a human comprising oral administration to said human of 300 mg of fenretinide once per day for three days, followed by oral administration of 200 mg of fenretinide for eleven days. In one embodiment the fenretinide is provided as LAU-7b.

In another aspect, the present invention provides for the use of fenretinide, fenretinide analog or pharmaceutically acceptable salt thereof for the preparation of a medicament for the treatment of a SARS-coronavirus infection in a human. In one embodiment the fenretinide, fenretinide analog or pharmaceutically acceptable salt thereof comprises 1 mg to 1000 mg of fenretinide. In a further embodiment the fenretinide, fenretinide analog or pharmaceutically acceptable salt thereof comprises 10 mg to 300 mg of fenretinide. In an alternative embodiment the fenretinide, fenretinide analog or pharmaceutically acceptable salt thereof results in a plasma concentration of a human of 0.5 μM to 10 μM of fenretinide. In a further embodiment the fenretinide, fenretinide analog or pharmaceutically acceptable salt thereof results in a plasma concentration of a human of 1 μM to 3 μM of fenretinide.

In another aspect, the present invention provides for a method of treating a SARS-coronavirus associated pneumonia in a human comprising administration to said human of a therapeutically effective amount of fenretinide, fenretinide analog or pharmaceutically acceptable salt thereof. In one embodiment the therapeutically effective amount of fenretinide, fenretinide analog or pharmaceutically acceptable salt thereof comprises 1 mg to 1000 mg of fenretinide. In further embodiment the therapeutically effective amount of fenretinide, fenretinide analog or pharmaceutically acceptable salt thereof comprises 10 mg to 300 mg of fenretinide. In an alternative embodiment the therapeutically effective amount of fenretinide, fenretinide analog or pharmaceutically acceptable salt thereof gives rise to a plasma concentration in said human of 0.5 μM to about 10 μM fenretinide. In a further embodiment the therapeutically effective amount of fenretinide, fenretinide analog or pharmaceutically acceptable salt thereof gives rise to a plasma concentration in said human of 1 μM to about 3 μM of fenretinide.

In another aspect, the present invention provides for the use of fenretinide, fenretinide analog or pharmaceutically acceptable salt thereof for the preparation of a medicament for the treatment of a SARS-coronavirus associated pneumonia in a human. In one embodiment the fenretinide, fenretinide analog or pharmaceutically acceptable salt thereof comprises 1 mg to 1000 mg of fenretinide. In a further embodiment the fenretinide, fenretinide analog or pharmaceutically acceptable salt thereof comprises 10 mg to 300 mg of fenretinide. In an alternative embodiment the fenretinide, fenretinide analog or pharmaceutically acceptable salt thereof results in a plasma concentration of a human of 0.5 μM to 10 μM of fenretinide. In a further embodiment the fenretinide, fenretinide analog or pharmaceutically acceptable salt thereof results in a plasma concentration of a human of 1 μM to 3 μM of fenretinide.

In another aspect, the present invention provides for a method of treating acute respiratory distress syndrome in a human comprising administration to said human of a therapeutically effective amount of fenretinide, fenretinide analog or pharmaceutically acceptable salt thereof. In one embodiment the therapeutically effective amount of fenretinide, fenretinide analog or pharmaceutically acceptable salt thereof comprises 1 mg to 1000 mg of fenretinide. In a further embodiment the therapeutically effective amount of fenretinide, fenretinide analog or pharmaceutically acceptable salt thereof comprises 10 mg to 300 mg of fenretinide. In an alternative embodiment the therapeutically effective amount of fenretinide, fenretinide analog or pharmaceutically acceptable salt thereof gives rise to a plasma concentration in said human of 0.5 μM to about 10 μM of fenretinide. In a further embodiment the therapeutically effective amount of fenretinide, fenretinide analog or pharmaceutically acceptable salt thereof gives rise to a plasma concentration in said human of 1 μM to about 3 μM of fenretinide.

In an alternative embodiment, the acute respiratory distress syndrome is associated with SARS-coronavirus. In a further embodiment the therapeutically effective amount of fenretinide, fenretinide analog or pharmaceutically acceptable salt thereof comprises 1 mg to 1000 mg of fenretinide. In a still further embodiment, the therapeutically effective amount of fenretinide, fenretinide analog or pharmaceutically acceptable salt thereof comprises 10 mg to 300 mg of fenretinide. In further embodiment the therapeutically effective amount of fenretinide, fenretinide analog or pharmaceutically acceptable salt thereof gives rise to a plasma concentration in said human of 0.5 μM to about 10 μM of fenretinide. In a still further embodiment, the therapeutically effective amount of fenretinide, fenretinide analog or pharmaceutically acceptable salt thereof gives rise to a plasma concentration in said human of 1 μM to about 3 μM of fenretinide.

In another aspect, the present invention provides for the use of fenretinide, fenretinide analog or pharmaceutically acceptable salt thereof for the preparation of a medicament for the treatment of acute respiratory distress syndrome in a human. In one embodiment the fenretinide, fenretinide analog or pharmaceutically acceptable salt thereof comprises 1 mg to 1000 mg of fenretinide. In a further embodiment the fenretinide, fenretinide analog or pharmaceutically acceptable salt thereof comprises 10 mg to 300 mg of fenretinide. In an alternative embodiment the fenretinide, fenretinide analog or pharmaceutically acceptable salt thereof results in a plasma concentration of fenretinide in a human of 0.5 μM to 10 μM. In a further embodiment the fenretinide, fenretinide analog or pharmaceutically acceptable salt thereof results in a plasma concentration of fenretinide in a human of 1 μM to 3 μM.

In an alternative embodiment, the acute respiratory distress syndrome is associated with SARS-coronavirus. In a further embodiment the fenretinide, fenretinide analog or pharmaceutically acceptable salt thereof comprises 1 mg to 1000 mg of fenretinide. In a still further embodiment, the fenretinide, fenretinide analog or pharmaceutically acceptable salt thereof comprises 10 mg to 300 mg of fenretinide. In a further embodiment the fenretinide, fenretinide analog or pharmaceutically acceptable salt thereof results in a plasma concentration of a human of 0.5 μM to 10 μM. In a still further embodiment, the fenretinide, fenretinide analog or pharmaceutically acceptable salt thereof results in a plasma concentration of a human of 1 μM to 3 μM.

In another aspect, the present invention provides a method of treating SARS-coronavirus infection in a human comprising administration to said human of a therapeutically effective amount of fenretinide, fenretinide analog or pharmaceutically acceptable salt thereof. In one embodiment, the therapeutically effective amount of fenretinide, fenretinide analog or pharmaceutically acceptable salt thereof comprises 1 mg to 1000 mg of fenretinide. In a further embodiment, the therapeutically effective amount of fenretinide, fenretinide analog or pharmaceutically acceptable salt thereof comprises 10 mg to 300 mg of fenretinide. In an alternative embodiment the therapeutically effective amount of fenretinide, fenretinide analog or pharmaceutically acceptable salt thereof gives rise to a plasma concentration of fenretinide in said human of 0.5 μM to about 10 μM. In a further embodiment the therapeutically effective amount of fenretinide, fenretinide analog or pharmaceutically acceptable salt thereof gives rise to a plasma concentration of fenretinide in said human of 1 μM to about 3 μM.

In another aspect, the present invention provides for the use of fenretinide, fenretinide analog or pharmaceutically acceptable salt thereof in the preparation of a medicament for the treatment of SARS-coronavirus infection in a human. In one embodiment the fenretinide, fenretinide analog or pharmaceutically acceptable salt thereof comprises 1 mg to 1000 mg fenretinide. In a further embodiment the fenretinide, fenretinide analog or pharmaceutically acceptable salt thereof comprises 10 mg to 300 mg of fenretinide. In an alternative embodiment the fenretinide, fenretinide analog or pharmaceutically acceptable salt thereof results in a plasma concentration of fenretinide in a human of 0.5 μM to 10 μM. In a further embodiment the fenretinide, fenretinide analog or pharmaceutically acceptable salt thereof results in a plasma concentration of fenretinide in a human of 1 μM to 3 μM.

In another aspect, the present invention provides a method of treating a SARS-coronavirus associated inflammation in a human comprising administration to said human of a therapeutically effective amount of fenretinide, fenretinide analog or pharmaceutically acceptable salt thereof. In one embodiment the therapeutically effective amount of fenretinide, fenretinide analog or pharmaceutically acceptable salt thereof comprises 1 mg to 1000 mg of fenretinide. In a further embodiment the therapeutically effective amount of fenretinide, fenretinide analog or pharmaceutically acceptable salt thereof comprises 10 mg to 300 mg of fenretinide. In an alternative embodiment the therapeutically effective amount of fenretinide, fenretinide analog or pharmaceutically acceptable salt thereof gives rise to a plasma concentration of fenretinide in said human of 0.5 μM to about 10 μM. In a further embodiment the therapeutically effective amount of fenretinide, fenretinide analog or pharmaceutically acceptable salt thereof gives rise to a plasma concentration of fenretinide in said human of 1 μM to about 3 μM.

In another aspect, the present invention provides for the use of fenretinide, fenretinide analog or pharmaceutically acceptable salt thereof for the preparation of a medicament for the treatment of a SARS-coronavirus associated inflammation in a human. In one embodiment the fenretinide, fenretinide analog or pharmaceutically acceptable salt thereof comprises 1 mg to 1000 mg of fenretinide. In a further embodiment the fenretinide, fenretinide analog or pharmaceutically acceptable salt thereof comprises 10 mg to 300 mg of fenretinide. In an alternative embodiment the fenretinide, fenretinide analog or pharmaceutically acceptable salt thereof results in a plasma concentration of fenretinide in a human of 0.5 μM to 10 μM. In a further embodiment the fenretinide, fenretinide analog or pharmaceutically acceptable salt thereof results in a plasma concentration of fenretinide in a human of 1 μM to 3 μM.

In another aspect, the present invention provides a method of prophylaxis of SARS-coronavirus infection in a human comprising administration to said human of a therapeutically effective amount of fenretinide, fenretinide analog or pharmaceutically acceptable salt thereof. In one embodiment the therapeutically effective amount of fenretinide, fenretinide analog or pharmaceutically acceptable salt thereof comprises 1 mg to 1000 mg of fenretinide. In a further embodiment the therapeutically effective amount of fenretinide, fenretinide analog or pharmaceutically acceptable salt thereof comprises 10 mg to 300 mg of fenretinide. In an alternative embodiment the therapeutically effective amount of fenretinide, fenretinide analog or pharmaceutically acceptable salt thereof gives rise to a plasma concentration of fenretinide in said human of 0.5 μM to about 10 μM. In a further embodiment the therapeutically effective amount of fenretinide, fenretinide analog or pharmaceutically acceptable salt thereof gives rise to a plasma concentration of fenretinide in said human of 1 μM to about 3 μM.

In another aspect, the present invention provides for the use of fenretinide, fenretinide analog or pharmaceutically acceptable salt thereof for the preparation of a medicament for the prophylaxis of a SARS-coronavirus infection in a human. In one embodiment the fenretinide, fenretinide analog or pharmaceutically acceptable salt thereof comprises 1 mg to 1000 mg of fenretinide. In a further embodiment the fenretinide, fenretinide analog or pharmaceutically acceptable salt thereof comprises 10 mg to 300 mg of LAU-7b. In an alternative embodiment the fenretinide, fenretinide analog or pharmaceutically acceptable salt thereof results in a plasma concentration of fenretinide in a human of 0.5 μM to 10 μM. In a further embodiment the fenretinide, fenretinide analog or pharmaceutically acceptable salt thereof results in a plasma concentration of fenretinide in a human of 1 μM to 3 μM.

In another aspect, the present invention provides a method of prophylaxis of SARS-coronavirus associated pneumonia in a human comprising administration to said human of a therapeutically effective amount of fenretinide, fenretinide analog or pharmaceutically acceptable salt thereof. In one embodiment the therapeutically effective amount of fenretinide, fenretinide analog or pharmaceutically acceptable salt thereof comprises 1 mg to 1000 mg of fenretinide. In a further embodiment the therapeutically effective amount of fenretinide, fenretinide analog or pharmaceutically acceptable salt thereof comprises 10 mg to 300 mg of fenretinide. In an alternative embodiment the therapeutically effective amount of fenretinide, fenretinide analog or pharmaceutically acceptable salt thereof gives rise to a plasma concentration of fenretinide in said human of 0.5 μM to about 10 μM. In a further embodiment the therapeutically effective amount of fenretinide, fenretinide analog or pharmaceutically acceptable salt thereof gives rise to a plasma concentration of fenretinide in said human of 1 μM to about 3 μM.

In another aspect, the present invention provides for the use of fenretinide, fenretinide analog or pharmaceutically acceptable salt thereof for the preparation of a medicament for the prophylaxis of a SARS-coronavirus associated pneumonia in a human. In one embodiment the fenretinide, fenretinide analog or pharmaceutically acceptable salt thereof comprises 1 mg to 1000 mg of fenretinide. In a further embodiment the fenretinide, fenretinide analog or pharmaceutically acceptable salt thereof comprises 10 mg to 300 mg of fenretinide. In an alternative embodiment the fenretinide, fenretinide analog or pharmaceutically acceptable salt thereof results in a plasma concentration of fenretinide in a human of 0.5 μM to 10 μM. In a further embodiment the fenretinide, fenretinide analog or pharmaceutically acceptable salt thereof results in a plasma concentration of fenretinide in a human of 1 μM to 3 μM.

In another aspect, the present invention provides a method of prophylaxis of acute respiratory distress syndrome in a human comprising administration to said human of a therapeutically effective amount of fenretinide, fenretinide analog or pharmaceutically acceptable salt thereof. In one embodiment the therapeutically effective amount of fenretinide, fenretinide analog or pharmaceutically acceptable salt thereof comprises 1 mg to 1000 mg of LAU-7b. In a further embodiment the therapeutically effective amount of fenretinide, fenretinide analog or pharmaceutically acceptable salt thereof comprises 10 mg to 300 mg of LAU-7b. In an alternative embodiment the therapeutically effective amount of fenretinide, fenretinide analog or pharmaceutically acceptable salt thereof gives rise to a plasma concentration of fenretinide in said human of 0.5 μM to about 10 μM. In a further embodiment the therapeutically effective amount of fenretinide, fenretinide analog or pharmaceutically acceptable salt thereof gives rise to a plasma concentration of fenretinide in said human of 1 μM to about 3 μM.

In an alternative embodiment the acute respiratory distress syndrome is associated with SARS-coronavirus. In further embodiment the therapeutically effective amount of fenretinide, fenretinide analog or pharmaceutically acceptable salt thereof comprises 1 mg to 1000 mg of fenretinide. In a still further embodiment, the therapeutically effective amount of fenretinide, fenretinide analog or pharmaceutically acceptable salt thereof comprises 10 mg to 300 mg of fenretinide. In a further embodiment the therapeutically effective amount of fenretinide, fenretinide analog or pharmaceutically acceptable salt thereof gives rise to a plasma concentration of fenretinide in said human of 0.5 μM to about 10 μM. In a still further embodiment, the therapeutically effective amount of fenretinide, fenretinide analog or pharmaceutically acceptable salt thereof gives rise to a plasma concentration of fenretinide in said human of 1 μM to about 3 μM.

In another aspect, the present invention provides for the use of fenretinide, fenretinide analog or pharmaceutically acceptable salt thereof for the preparation of a medicament for the prophylaxis of acute respiratory distress syndrome in a human. In one embodiment the fenretinide, fenretinide analog or pharmaceutically acceptable salt thereof comprises 1 mg to 1000 mg of fenretinide. In a further embodiment the fenretinide, fenretinide analog or pharmaceutically acceptable salt thereof comprises 10 mg to 300 mg of fenretinide. In an alternative embodiment the fenretinide, fenretinide analog or pharmaceutically acceptable salt thereof results in a plasma concentration of fenretinide in a human of 0.5 μM to 10 μM. In a further embodiment the fenretinide, fenretinide analog or pharmaceutically acceptable salt thereof results in a plasma concentration of fenretinide in a human of 1 μM to 3 μM.

In an alternative embodiment, the acute respiratory distress syndrome is associated with SARS-coronavirus. In a further embodiment the fenretinide, fenretinide analog or pharmaceutically acceptable salt thereof comprises 1 mg to 1000 mg of fenretinide. In a still further embodiment, the fenretinide, fenretinide analog or pharmaceutically acceptable salt thereof comprises 10 mg to 300 mg of fenretinide. In a further embodiment the fenretinide, fenretinide analog or pharmaceutically acceptable salt thereof results in a plasma concentration of fenretinide in a human of 0.5 μM to 10 μM. In a still further embodiment, the fenretinide, fenretinide analog or pharmaceutically acceptable salt thereof results in a plasma concentration of fenretinide in a human of 1 μM to 3 μM.

In one aspect, the present invention provides for a method of treating hypoxemia in a human comprising administration to said human of a therapeutically effective amount of fenretinide, fenretinide analog or pharmaceutically acceptable salt thereof. In one embodiment, the therapeutically effective amount of fenretinide, fenretinide analog or pharmaceutically acceptable salt thereof comprises 1 mg to 1000 mg of fenretinide. In a further embodiment, the therapeutically effective amount of fenretinide, fenretinide analog or pharmaceutically acceptable salt thereof comprises 10 mg to 300 mg of fenretinide. In an alternative embodiment the therapeutically effective amount of fenretinide, fenretinide analog or pharmaceutically acceptable salt thereof gives rise to a plasma concentration in said human of 0.5 μM to about 10 μM of fenretinide. In a further embodiment the therapeutically effective amount of fenretinide, fenretinide analog or pharmaceutically acceptable salt thereof gives rise to a plasma concentration in said human of 1 μM to about 3 μM of fenretinide. In one embodiment the therapeutically effective amount of fenretinide, fenretinide analog or pharmaceutically acceptable salt thereof comprises an inhaled dosage form of fenretinide administered to the lungs of said human until about 1.8 μg/kg to about 3.6 μg/kg of fenretinide is delivered to the lungs. In one embodiment the hypoxemia arises from, or is a complication of, acute respiratory distress syndrome.

In another aspect, the present invention provides for the use of fenretinide, fenretinide analog or pharmaceutically acceptable salt thereof for the preparation of a medicament for the treatment of hypoxemia in a human. In one embodiment the fenretinide, fenretinide analog or pharmaceutically acceptable salt thereof comprises 1 mg to 1000 mg of fenretinide. In a further embodiment the fenretinide, fenretinide analog or pharmaceutically acceptable salt thereof comprises 10 mg to 300 mg of fenretinide. In an alternative embodiment the fenretinide, fenretinide analog or pharmaceutically acceptable salt thereof results in a plasma concentration of a human of 0.5 μM to 10 μM of fenretinide. In a further embodiment the fenretinide, fenretinide analog or pharmaceutically acceptable salt thereof results in a plasma concentration of a human of 1 μM to 3 μM of fenretinide. In one embodiment the fenretinide, fenretinide analog or pharmaceutically acceptable salt thereof comprises an inhaled dosage form of fenretinide capable of being administered to the lungs of said human until about 1.8 μg/kg to about 3.6 μg/kg of fenretinide is delivered to the lungs. In one embodiment the hypoxemia arises from, or is a complication of, acute respiratory distress syndrome.

In one aspect, the present invention provides for a method of prophylaxis of hypoxemia in a human comprising administration to said human of a therapeutically effective amount of fenretinide, fenretinide analog or pharmaceutically acceptable salt thereof. In one embodiment, the therapeutically effective amount of fenretinide, fenretinide analog or pharmaceutically acceptable salt thereof comprises 1 mg to 1000 mg of fenretinide. In a further embodiment, the therapeutically effective amount of fenretinide, fenretinide analog or pharmaceutically acceptable salt thereof comprises 10 mg to 300 mg of fenretinide. In an alternative embodiment the therapeutically effective amount of fenretinide, fenretinide analog or pharmaceutically acceptable salt thereof gives rise to a plasma concentration in said human of 0.5 μM to about 10 μM of fenretinide. In a further embodiment the therapeutically effective amount of fenretinide, fenretinide analog or pharmaceutically acceptable salt thereof gives rise to a plasma concentration in said human of 1 μM to about 3 μM of fenretinide. In one embodiment the therapeutically effective amount of fenretinide, fenretinide analog or pharmaceutically acceptable salt thereof comprises an inhaled dosage form of fenretinide administered to the lungs of said human until about 1.8 μg/kg to about 3.6 μg/kg of fenretinide is delivered to the lungs. In one embodiment the hypoxemia arises from, or is a complication of, acute respiratory distress syndrome.

In another aspect, the present invention provides for the use of fenretinide, fenretinide analog or pharmaceutically acceptable salt thereof for the preparation of a medicament for the prophylaxis of hypoxemia in a human. In one embodiment the fenretinide, fenretinide analog or pharmaceutically acceptable salt thereof comprises 1 mg to 1000 mg of fenretinide. In a further embodiment the fenretinide, fenretinide analog or pharmaceutically acceptable salt thereof comprises 10 mg to 300 mg of fenretinide. In an alternative embodiment the fenretinide, fenretinide analog or pharmaceutically acceptable salt thereof results in a plasma concentration of a human of 0.5 μM to 10 μM of fenretinide. In a further embodiment the fenretinide, fenretinide analog or pharmaceutically acceptable salt thereof results in a plasma concentration of a human of 1 μM to 3 μM of fenretinide. In one embodiment the fenretinide, fenretinide analog or pharmaceutically acceptable salt thereof comprises an inhaled dosage form of fenretinide capable of being administered to the lungs of said human until about 1.8 μg/kg to about 3.6 μg/kg of fenretinide is delivered to the lungs. In one embodiment the hypoxemia arises from, or is a complication of, acute respiratory distress syndrome.

In another aspect, the present invention provides a method of treating SARS-coronavirus infection in a human comprising administration to said human of a therapeutically effective amount of fenretinide, fenretinide analog or pharmaceutically acceptable salt thereof in combination with a therapeutic amount of a delayed chain terminator antiviral compound. In one embodiment the delayed chain terminator antiviral compound is selected from the group comprising remdesivir, penciclovir, cidofovir and entecavir. In a further embodiment, the therapeutically effective amount of fenretinide, fenretinide analog or pharmaceutically acceptable salt thereof gives rise to a plasma concentration of fenretinide in said human of 0.5 μM to about 10 μM. In a further embodiment the therapeutically effective amount of fenretinide, fenretinide analog or pharmaceutically acceptable salt thereof gives rise to a plasma concentration of fenretinide in said human of 1.5 μM to about 3 μM.

In another aspect, the present invention provides for the use of fenretinide, fenretinide analog or pharmaceutically acceptable salt thereof in combination with a delayed chain terminator antiviral compound in the preparation of a medicament for the treatment of SARS-coronavirus infection in a human. In one embodiment the delayed chain terminator antiviral compound is selected from the group comprising remdesivir, penciclovir, cidofovir and entecavir. In a further embodiment the fenretinide, fenretinide analog or pharmaceutically acceptable salt thereof results in a plasma concentration of fenretinide in a human of 0.5 μM to 10 μM. In a further embodiment the fenretinide, fenretinide analog or pharmaceutically acceptable salt thereof results in a plasma concentration of fenretinide in a human of 1.5 μM to 3 μM.

### BRIEF DESCRIPTION OF THE FIGURES

FIG. 1 shows a linear regression curve for fenretinide anti-viral effect on SARS-CoV-2 in Vero E6 cells;

FIG. 2 shows the effect of fenretinide on physiological parameters in LPS induced ARDS in mice after 24 hours;

FIG. 3. shows the effect of fenretinide on neutrophils in (A) BALF and (B) blood in

LPS induced ARDS in mice after 24 hours;

FIG. 4. shows the effect of fenretinide on physiological parameters in LPS induced ARDS in mice after 72 hours;

FIG. 5 shows the effect of fenretinide on the pulmonary congestion index in LPS induced ARDS in mice after 72 hours;

FIG. 6 shows the effect of fenretinide on BALF cell count (A-D) in LPS induced ARDS in mice after 72 hours;

FIG. 7shows the effect of fenretinide on lung weight (A), lung protein (B-C) and BALF protein content (D-E) in LPS induced ARDS in mice after 72 hours;

FIG. 8 shows the histopathological assessment of lung injury in LPS induced ARDS in mice treated with fenretinide, after 72 hours;

FIG. 9 shows oxygen saturation in an LPS induced ARDS model of mice, when treated with inhaled fenretinide;

FIG. 10 shows blood reticulocyte counts in LPS induced ARDS model of mice, when treated with inhaled or orally administered fenretinide; and

FIG. 11 shows myeloperoxidase activity in the BALF (A) and lung protein concentration (B) in LPS induced mouse model of ARDS, when treated with inhaled fenretinide.

### DETAILED DESCRIPTION OF THE PRESENT INVENTION

The present invention provides for novel methods and compositions useful for the treatment of SARS-coronavirus infection, SARS-coronavirus associated pneumonia, ARDS, ARDS associated hypoxemia and pneumonia induced ARDS.

In one aspect of the present invention, “co-administered” and “co-administration” as relating to a patient, refer to administering to the subject a compound and/or composition of the present invention, or salt thereof, along with a compound and/or composition that may also treat any of the diseases or disorders contemplated within the invention. In one embodiment, the co-administered compounds and/or compositions are administered separately, or in any kind of combination as part of a single therapeutic approach. The co-administered compound and/or composition may be formulated in any kind of combination as mixtures of solids and liquids under a variety of solid, gel, and liquid formulations, and as a solution.

As used herein, the term “about” will be understood by one skilled in the art to vary to some extent by the context under which it is used. As used herein, when referring to a measurable value such as an amount, time duration, and the like; the term “about” shall encompass variations of +/−20%, or +/−10%, more preferably +/−5%, even more preferably +/−1%, and still more preferably +/−0.1% from the specified value, as such variations are appropriate to perform the disclosed methods.

As used herein, “administration”, means providing a compound and/or composition of the present invention to a subject by any suitable method.

As used herein, “alkyl”, by itself or as part of another substituent, means, unless otherwise stated, a straight or branched chain hydrocarbon having the number of carbon atoms designated and includes straight, branched chain, or cyclic substituent groups. Examples include methyl, ethyl, propyl, isopropyl, butyl, isobutyl, tert-butyls, pentyl, neopentyl, hexyl and cyclopropylmethyl.

As used herein, “ameliorate” means to decrease, suppress, attenuate, diminish, arrest, or stabilize the development or progression of a disease or disorder.

As used herein, an “amorphous solid dispersion” means a dispersion in which at least a major portion (i.e., more than 50%) of the fenretinide, fenretinide analog, or salt thereof in the dispersion is in amorphous form. By “amorphous” is meant that the fenretinide, fenretinide analog, or salt thereof is in a non-crystalline state. In embodiments, at least 55, 60, 65, 70, 75, 80, 85, 90% or 95% of the fenretinide, fenretinide analog, or salt thereof (by weight) in the dispersion is in the amorphous form.

As used herein, the term “composition” or “pharmaceutical composition” refers to a mixture of at least one compound useful within the invention with a pharmaceutically acceptable carrier. The pharmaceutical composition facilitates administration of the compound to a subject.

As used herein, an “effective amount” means the amount of a compound that is required to ameliorate the symptom of a disease, prevent the worsening of the disease, or reduce viral load, as appropriate, relative to an untreated patient. The effective amount of active compound(s) used to practise the present invention for therapeutic treatment of a disease varies depending upon the manner of administration, the age, body weight, and general health of the subject. Ultimately, the attending physician will decide the appropriate amount and dosage regimen. Such amount is therefore referred to as an “effective amount”.

As used herein, “excipient” has its normal meaning in the art and is any ingredient that is not an active ingredient (drug) itself. Excipients include for example binders, lubricants, diluents, fillers, thickening agents, disintegrants, plasticizers, coatings, barrier layer formulations, lubricants, stabilizing agent, release-delaying agents and other components. “Pharmaceutically acceptable excipient” as used herein refers to any excipient that does not interfere with effectiveness of the biological activity of the active ingredients and that is not toxic to the subject, i.e., is a type of excipient and/or is for use in an amount which is not toxic to the subject. Excipients are well known in the art, and the present system is not limited in these respects. In certain embodiments, the composition includes excipients, including for example and without limitation, one or more binders (binding agents), thickening agents, surfactants, diluents, release-delaying agents, colorants, flavoring agents, fillers, disintegrants/dissolution promoting agents, lubricants, plasticizers, silica flow conditioners, glidants, anti-caking agents, anti-tacking agents, stabilizing agents, anti-static agents, swelling agents and any combinations thereof. As those of skill would recognize, a single excipient can fulfill more than two functions at once, e.g., can act as both a binding agent and a thickening agent. As those of skill will also recognize, these terms are not necessarily mutually exclusive.

As used herein, “pharmaceutically acceptable” means a material, such as a carrier or diluent, which does not abrogate the biological activity or properties of the compound useful within the present invention and is relatively non-toxic. It is intended that “pharmaceutically acceptable” materials may be administered to a subject without causing undesirable biological effects or interacting in a deleterious manner with any of the components of the composition in which it is contained.

As used herein, “pharmaceutically acceptable salt” means a salt of the administered compounds prepared from pharmaceutically acceptable non-toxic acids, including inorganic acids, organic acids, inorganic bases, organic bases, solvates, hydrates, or clathrates thereof. The compounds described herein may form salts with acids or bases, and such salts are included in the present invention. In one embodiment, the salts are pharmaceutically acceptable salt. The term “salts” includes addition of free acids or bases that are useful within the methods of the present invention. The term “pharmaceutically acceptable salt” refers to salts that possess toxicity profiles within a range that affords utility in pharmaceutical and disease and disorder treatment of patient applications. Pharmaceutically unacceptable salts may nonetheless possess properties which have utility in the practise of the present invention, and one skilled in the art would be capable of identifying and using a pharmaceutically unacceptable salt as part of the treatment of a disease or disorder of patients, as contemplated herein, or as part of the manufacturing of a compound of the present invention.

As used herein, “solid dispersion” means a solid material, in which a drug (e.g., fenretinide) is dispersed in the solid matrix polymer. Such solid dispersions are also referred to in the art as “molecular dispersions” or “solid solutions” of the drug in the polymer. Solid dispersions may be obtained by various techniques, for example fast evaporation, spray-drying, precipitation or melt extrusion (e.g., hot melt extrusion,

HME). In an embodiment, the solid dispersion is obtained by spray-drying (spray-dried solid dispersion).

As used herein, “LAU-7b” means an improved oral formulation of fenretinide, formulated as spray dried solid amorphous dispersion suitable for encapsulation, which contains LAU-7b SDI in addition to inert excipients in external phase to help flowability for encapsulation, and ascorbic acid for increased stability. LAU-7b SDI is a spray dry intermediate of LAU-7b, with each 2.5mg of LAU-7b-SDI containing 1 mg fenretinide, 1.49 mg povidone, 0.006 mg butylated-hydroxyanisole, and 0.004 mg butylated hydroxytoluene.

### Fenretinide and Analogs Thereof

Fenretinide (4-hydroxyphenyl retinamide; also referred to as 4-HPR, which has CAS registry number 65646-68-6, is a synthetic retinoid of the following formula II:

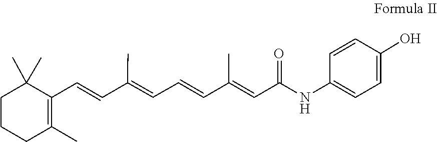 [SMILES: CC1=C(/C=C/C(C)=C/C=C/C(C)=C/C(=O)Nc2ccc(O)cc2)C(C)(C)CCC1]

Functional analogs (and/or metabolites) of fenretinide (i.e., which exhibit the same biological activity as fenretinide) may also be used according to the present disclosure. As used herein, a “fenretinide analog” refers to a compound that shares certain chemical structural features with fenretinide but at the same time comprises one or more modifications thereto, and which exhibits similar biological activity as fenretinide (but may exhibit such activity to a different extent). Examples of analogs of fenretinide that may be used include, but are not limited to, 4-oxo-N-(4-hydroxyphenyl)retinamide (4-oxo-4-HPR), N-(4-methoxyphenyl)retinamide (4-MPR), 4-Hydroxybenzylretinone, C-glycoside and arylamide analogues of N-(4-hydroxyphenyl) retinamide-O-glucuronide, including but not limited to 4-(retinamido)phenyl-C-glucuronide, 4-(retinamido)phenyl-C-glucoside, 4-(retinamido)benzyl-C-xyloside; and retinoyl β-glucuronide analogues such as, for example, 1-(β-D-glucopyranosyl) retinamide, 1-(D-glucopyranosyluronosyl) retinamide and bexarotene, described in WO 07/136636, U.S. Patent Application No. 2006/0264514, U.S. Pat. Nos. 5,516,792, 5,663,377, 5,599,953, 5,574,177, Anding et al. (Anding, A. L. et al., Cancer Research, https://doi.org/10.1158/0008-5472.CAN-07-0727, 2007) and Bhatnagar et al. (Bhatnagar, R., et al., Biochemical Pharmacology, https://doi.org/10.1016/0006-2952(91)90563-K, 1991). In an embodiment, the fenretinide/fenretinide analog is represented by formula I:

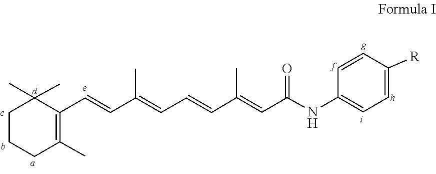

R is OH, COOH, CH₂OH, CH₂CH₂OH, or CH₂COOH; carbons a-d and f-i are optionally substituted with one or more groups selected from CH₃, OH, COOH, (CH₃)₂ and CH₂OH, or any combination thereof, and carbon e is optionally substituted with a C1-C3 alkyl group that is optionally substituted with CH₃ and/or OH.

Any salts of fenretinide or fenretinide analogs may also be used in the method or use described herein.

The method or use comprises the administration or use of fenretinide or an analog of fenretinide, or a pharmaceutically acceptable salt thereof.

Fenretinide is a small molecule synthetic retinoid derivative, with well-documented history of safety in non-clinical and clinical studies. Initially explored for prevention and treatment of cancer, fenretinide was also studied for non-oncological indications such as age-related macular degeneration.

### Dosage

Any suitable amount of fenretinide, fenretinide analog or salt thereof may be administered to a subject. The dosages will depend on many factors including the mode of administration. Typically, the amount of fenretinide, fenretinide analog or salt thereof, contained within a single dose will be an amount that effectively prevents, delays or treats the SARS-coronavirus associated pneumonia without inducing significant toxicity.

For prophylaxis, treatment or reduction in the severity of SARS-coronavirus infection, the appropriate dosage of the compound/composition depends on the severity of the pneumonia, whether the compound/composition is administered for preventive or therapeutic purposes, previous or concomitant therapy, the patient's clinical history and response to the compound/composition, and the discretion of the attending physician. The fenretinide, fenretinide analog or salt thereof, is/are suitably administered to the patient at one time or over a series of treatments.

The present invention provides dosages for the compounds and compositions comprising same. For example, depending on the severity of the disease, the effective dose may be 0.5 mg/kg, 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, and up to 100 mg/kg of fenretinide. A typical daily dosage might range from about 1mg/kg to 20 mg/kg or more, depending on the factors mentioned above; provided by way of administration to a patient of fenretinide, fenretinide analog or salt thereof that is administered in an amount of 1 mg to about 1000 mg, preferably about 10 mg to 300 mg. For repeated administrations over several days or longer, the treatment is sustained until the desired suppression of disease symptoms occurs. The present invention contemplates establishing a plasma concentration in the patient of fenretinide, fenretinide analog or salt thereof of about 0.5 μM to about 10 μM, preferably of about 1 μM to about 3 μM.

However, other dosage regimens may be useful. The progress of this therapy is easily monitored by conventional techniques and assays. These are simply guidelines since the actual dose must be carefully selected and titrated by the attending physician based upon clinical factors unique to each patient or by a nutritionist. The optimal daily dose will be determined by methods known in the art and will be influenced by factors such as the age of the patient and other clinically relevant factors. In addition, patients may be taking medications for other diseases or conditions. The other medications may be continued during the time that fenretinide, fenretinide analog or salt thereof, is given to the patient, but it is particularly advisable in such cases to begin with lower doses to determine if adverse side effects are experienced.

### Compositions

The fenretinide, fenretinide analog or salt thereof, may be combined with one or more optional carriers or excipients to formulate the compound(s) into suitable dosage formulations, such as tablets, capsules (e.g., hard gelatine capsules), caplets, suspensions, powders for suspensions, and the like. Such compositions may be prepared by mixing the active ingredient (e.g., fenretinide) having the desired degree of purity; with one or more optional pharmaceutically acceptable carriers, excipients and/or stabilizers in a manner well known in the pharmaceutical art. Supplementary active compounds can also be incorporated into the compositions. The carrier/excipient can be suitable, for example, for oral, intravenous, parenteral, subcutaneous, intramuscular, intranasal or pulmonary (e.g., aerosol) administration (see Remington: The Science and Practice of Pharmacy, by Loyd V Allen, Jr, 2012, 22nd edition, Pharmaceutical Press; Handbook of Pharmaceutical Excipients, by Rowe et al., 2012, 7th edition, Pharmaceutical Press). Therapeutic formulations are prepared using standard methods known in the art.

Examples of matrix materials, fillers, or diluents include, without limitation, lactose, mannitol, xylitol, microcrystalline cellulose, dibasic calcium phosphate (anhydrous and dihydrate), starch, and any combination thereof.

Examples of disintegrants include, without limitation, sodium starch glycolate, sodium alginate, carboxy methyl cellulose sodium, methyl cellulose, and croscarmellose sodium, and crosslinked forms of polyvinyl pyrrolidone such as those sold under the trade name CROSPOVIDONE® (available from BASF Corporation), and any combination thereof.

Examples of binders include, without limitation, methyl cellulose, microcrystalline cellulose, starch, and gums such as guar gum, tragacanth, and any combination thereof.

Examples of lubricants include, without limitation, magnesium stearate, calcium stearate, stearic acid, and any combination thereof.

Examples of glidants include, without limitation, metal silicates, silicon dioxides, higher fatty acid metal salts, metal oxides, alkaline earth metal salts, and metal hydroxides. Examples of preservatives include, without limitation, sulfites (an antioxidant), benzalkonium chloride, methyl paraben, propyl paraben, benzyl alcohol, sodium benzoate, and any combination thereof.

Examples of suspending agents or thickeners, without limitation, include xanthan gum, starch, guar gum, sodium alginate, carboxymethyl cellulose, sodium carboxymethyl cellulose, methyl cellulose, hydroxypropyl methyl cellulose, polyacrylic acid, silica gel, aluminum silicate, magnesium silicate, titanium dioxide, and any combination thereof.

Examples of anti-caking agents or fillers, without limitation, include silicon oxide, lactose, and any combination thereof.

Examples of solubilizers include, without limitation, ethanol, propylene glycol, polyethylene glycol, and any combination thereof.

Examples of antioxidants include, without limitation, phenolic-based antioxidants such as butylated hydroxyanisole (BHA), butylated hydroxytoluene (BHT), tert-butyl-hydroquinone (TBHQ), 4-hydroxymethyl-2,6-di-tert-butylphenol (HMBP), 2,4,5-trihydroxy-butyrophenone (THBP), propyl gallate (PG), triamyl gallate, gallic acid (GA), α-Tocopherol (vitamin E), tocopherol acetate, reducing agents such as L-ascorbic acid (vitamin C), L-ascorbyl palmitate, L-ascorbyl stearate, thioglycolic acid

(TGA), ascorbyl palmitate (ASP), sulphite-based antioxidants such as sodium sulphite, sodium metabisulphite, sodium bisulphite and thioglycerol and other agents such as disodium ethylenediamine tetraacetate (EDTA), sodium pyrophosphate, sodium metaphosphate, methionine, erythorbic acid and lecithin, and any combination thereof. In an embodiment, the formulation comprises a combination of antioxidants. In an embodiment, the formulation comprises a combination of BHA and BHT. In an embodiment, the formulation comprises ascorbic acid.

Another class of excipients is surfactants, optionally present from about 0 to about 10 wt %. Suitable surfactants include, without limitation, fatty acid and alkyl sulfonates; commercial surfactants such as benzalkonium chloride (HYAMINE® 1622, available from Lonza, Inc., Fairlawn, N.J.); dioctyl sodium sulfosuccinate (DOCUSATE SODIUM, available from Mallinckrodt Spec. Chem., St. Louis, Mo.); polyoxyethylene sorbitan fatty acid esters (TWEEN®, available from ICI Americas Inc., Wilmington, Del.; LIPOSORB® O-20, available from Lipochem Inc., Patterson N.J.; CAPMUL™ POE-0, available from Abitec Corp., Janesville, Wis.); and natural surfactants such as sodium taurocholic acid, 1-palm itoyl-2-oleoyl-sn-glycero-3-phosphocholine, lecithin, and other phospholipids and mono- and diglycerides, and any combination thereof. Such materials can be employed to increase the rate of dissolution by, for example, facilitating wetting, or otherwise increase the rate of drug release from the dosage form.

Other conventional excipients, including pigments, lubricants, flavorants, humectants, solution retarding agents, absorption accelerators, wetting agents, absorbents, and other ones well-known in the art, may be employed in the compositions of this invention. For example, excipients such as pigments, lubricants, flavorants, and so forth may be used for customary purposes and in typical amounts without adversely affecting the properties of the compositions.

Other components commonly added to pharmaceutical compositions include, e.g., inorganic salts such as sodium chloride, potassium chloride, calcium chloride, sodium phosphate, potassium phosphate, sodium bicarbonate; and organic salts such as sodium citrate, potassium citrate, sodium acetate, etc.

In an embodiment, the fenretinide, fenretinide analog or salt thereof is present in the composition as an amorphous solid dispersion as described in U.S. Patent Publication No. 2017/0189356 A1, which is incorporated by reference in its entirety.

Examples of “matrix polymers”, also referred to in the field as “concentration-enhancing polymers” or “dispersion polymers”, which may be suitable for use in the present invention, are discussed in detail in for example U.S. Pat. Nos. 7,780,988 and 7,887,840. The matrix polymer can be any pharmaceutically acceptable polymer that, once co-processed with the fenretinide, fenretinide analog, or salt thereof, functions to maintain the fenretinide/fenretinide analog in amorphous form.

Examples of polymers that may be suitable for use with the present invention comprise non-ionizable (neutral) non-cellulosic polymers. Exemplary polymers include: vinyl polymers and copolymers having at least one substituent selected from hydroxyl, alkylacyloxy, and cyclicamido; polyvinyl alcohols that have at least a portion of their repeat units in the unhydrolyzed (vinyl acetate) form; polyvinyl alcohol polyvinyl acetate copolymers; polyvinyl pyrrolidone; and polyethylene polyvinyl alcohol copolymers; and polyoxyethylene-polyoxypropylene copolymers.

Other examples of polymers that may be suitable for use with the present invention comprise ionizable non-cellulosic polymers. Exemplary polymers include: carboxylic acid-functionalized vinyl polymers, such as the carboxylic acid functionalized polymethacrylates and carboxylic acid functionalized polyacrylates such as the EUDRAGIT® series, amine-functionalized polyacrylates and polymethacrylates; proteins such as gelatin and albumin; and carboxylic acid functionalized starches such as starch glycolate.

Other examples polymers that may be suitable for use with the present invention comprise nonionizable cellulosic polymers that may be used as the polymer include: hydroxypropyl methyl cellulose acetate, hydroxypropyl methyl cellulose (HPMC), hydroxypropyl cellulose, methyl cellulose, hydroxyethyl methyl cellulose, hydroxyethyl cellulose acetate, hydroxyethyl ethyl cellulose, and the like.

While specific polymers have been discussed as being suitable for use in the dispersions formable by the present invention, blends of such polymers may also be suitable. Thus, the term “matrix polymer” is intended to include blends of polymers in addition to a single species of polymer.

In an embodiment, the matrix polymer comprises polyvinylpyrrolidone. In another embodiment, the matrix polymer is a polyvinylpyrrolidone, for example polymers sold under the trade-name Plasdone® (povidones), polyvinylpyrrolidone K12, polyvinylpyrrolidone K17, polyvinylpyrrolidone K25, polyvinylpyrrolidone K30 or polyvinylpyrrolidone K90.

In an embodiment, the ratio of the fenretinide, fenretinide analog, or salt thereof/matrix polymer is from about 1:5 to about 5:1, in further embodiments about 1:4 to about 4:1, about 1:3 to about 3:1, about 1:2 to about 2:1 or about 1.5:1 to about 1:1.5, by weight. In an embodiment, the solid dispersion comprises between about 30 to about 50% of the fenretinide, fenretinide analog, or salt thereof, and between about 50 to about 70% of matrix polymer. In another embodiment, the solid dispersion comprises between about 40% of the fenretinide, fenretinide analog, or salt thereof, and about 60% of matrix polymer, by weight.

In an embodiment, the solid dispersion comprises one or more additives. Additives that may be suitable for use with the present invention comprise antioxidant agents. Exemplary antioxidants include: L-ascorbic acid (vitamin C), propyl gallate, sodium sulfite, sodium metabisulfite, sodium bisulfite, thioglycerol, thioglycollic acid, tocopherols and tocotrienols, butylated hydroxyanisole (BHA), butylated hydroxytoluene (BHT) or any combination thereof. In an embodiment, the matrix polymer or solid dispersion comprises BHA and/or BHT as antioxidant agent(s). In an embodiment, the matrix polymer or solid dispersion comprises BHA and BHT as antioxidant agents. In an embodiment, the matrix polymer comprises L-ascorbic acid as antioxidant agent. In an embodiment, the antioxidant agent(s) is/are present in an amount of about 0.01% to about 5%, in further embodiments in an amount of about 0.1% to about 5%, about 0.2% to about 4%, 0.5% to about 3% or 0.5% to about 2%.

The amorphous solid dispersion of fenretinide, fenretinide analog, or salt thereof may be combined with one or more optional excipients as described above.

In an embodiment, the amorphous solid dispersion of fenretinide, fenretinide analog, or salt thereof is combined with a disintegrant, for example a cross-linked sodium carboxymethylcellulose e.g., croscarmellose (Solutab®). Other examples of disintegrants include corn starch, potato starch, sodium carboxymethylcellulose, sodium starch glycolate, sodium croscarmellose, crospovidone, and any combination thereof. In an embodiment, the disintegrant is present in an amount from about 2% to about 10% by weight, for example from about 3% to about 8% or about 4% to about 6% by weight.

In an embodiment, the amorphous solid dispersion of fenretinide, fenretinide analog, or salt thereof is combined with a lubricant, for example magnesium stearate. Other examples of lubricants include talc, silicon dioxide, stearic acid, and sodium stearyl fumarate. In an embodiment, the lubricant is present in an amount from about 0.5 to about 2% by weight, for example from about 0.8 to about 1.2% or about 1% by weight.

In an embodiment, the amorphous solid dispersion of fenretinide, fenretinide analog, or 30 salt thereof is combined with a filler or diluent, for example microcrystalline cellulose (Avicel®, such as Avicel®PH-102) and/or calcium hydrogen phosphate dehydrate (Encompress®). Other examples of fillers or diluents include crystalline cellulose, cellulose derivatives, acacia, corn starch, lactose, mannitol, sugars, calcium phosphate, calcium carbonate, gelatins, and any combination thereof. In an embodiment, the filler or diluent is present in an amount from about 35 20 to about 45% by weight, for example from about 30% to about 40% by weight, e.g., about 35%.

In an embodiment, the amorphous solid dispersion of fenretinide, fenretinide analog, or salt thereof is combined one or more antioxidants, for example butylated hydroxyanisole (BHA), butylated hydroxytoluene (BHT), citric acid, sodium metabisulfite, alpha-tocopherol and/or L-ascorbic acid.

In certain embodiments, the amorphous solid dispersion as disclosed herein is formulated as an oral dosage formulation. Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an elixir or syrup, or as pastilles (using an inert matrix, such as gelatin and glycerin, or sucrose and acacia), and the like, each containing a predetermined amount of an active ingredient. A composition may also be administered as a bolus, electuary, or paste.

In an embodiment, the oral dosage formulation is a tablet. A tablet may be made by compression or molding, optionally with one or more accessory ingredients. Compressed tablets may be prepared using binder, lubricant, inert diluent, preservative, disintegrant, surface-active or dispersing agent. Molded tablets may be made by molding in a suitable machine a mixture of the powdered inhibitor(s) moistened with an inert liquid diluent.

In some embodiments of the oral dosage formulation as disclosed herein, the amorphous solid dispersion is present in an amount of from about 10 to about 90%, about 20 to about 80%, about 30 to about 60% or about 45 to about 55% by weight, or another range within the values provided herein.

The Role of Membrane Lipids and cPLA2α on Coronavirus Attachment, Entry and Replication.

Viruses are obligatory intracellular parasites; they must enter host cells before they can initiate their life cycle. The entry of SARS-coronavirus into cells can occur via direct membrane fusion between the virus and plasma membrane, or by taking advantage of cell's endocytic machinery. Direct membrane fusion at the cell surface is pH-independent, while entry via the endocytic pathway usually depends on the low pH of endocytic vesicles involving angiotensin-converting enzyme 2 (ACE2), the functional receptor of SARS-coronavirus, from the cell surface to endosomes. Wang et al. also showed that the endocytic virus entry also involves cholesterol- and sphingolipid-rich lipid raft microdomains in the plasma membrane, which have been shown to act as platforms for many physiological signaling pathways (Wang, H. et al., Cell Research, https://doi.org/10.1038/cr.2008.15, 2008).

After entering the cell and uncoating, the virus induces rearrangement of the cellular membrane lipids to form double-membrane vesicles (DMVs), where the coronavirus replication transcription complex (RTC) is assembled and anchored. Coronavirus also forms large replicative organelles (ROs) that are thought to provide a structural scaffold for the viral RNA synthesis. Given the major membrane rearrangements occurring in virus-infected cells, enzymes involved in cellular lipid metabolism have been suggested to play a major role in this process. Muller et al., reported essential role for cytosolic phospholipase A2α0 (cPLA2α) in the production of DMV-associated coronaviral viral replication/transcription complexes. It was described that cPLA2α is involved in generating certain free fatty acids and lysophospholipids and its activity is modulated, at least in part, by mitogen-activated protein kinase (MAPK). (Muller, C. et al., Journal of Virology, https://doi.org/10.1128/JVI.01463-17. 2018) It was also shown that the pharmacological inhibition of cPLA2α, drastically reduces coronavirus RNA synthesis and, as a consequence, protein accumulation and the production of infectious virus progeny. The data suggest that the inhibition of cPLA2α activity blocks an early step in the viral replication cycle, most likely the formation of virus-induced ROs.

More recently, Fernandez-Oliva et al., conducted an extensive review of the role played by membrane lipid composition in viral and bacterial infections and concluded that therapeutic approaches based on specific factors of host—pathogen interactions involving membrane lipids are a promising avenue to overcome treatment failure in infectious diseases. Because many viruses and bacteria use lipids to build neo-organelles for replication and persistence, compounds that interfere with host lipid synthesis, transport, and signalling pathways may become efficient antivirals or antibiotics. (Fernández-Oliva A., et al., Cellular Microbiology, https://doi. org/10.1111/cm i.12996, 2019).

### The Role of ERK/MAPK and NF-kB Signalling Pathways.

When the cells are exposed to viruses, apoptosis and immune responses are induced as a form of host defence. Apoptosis is induced as one of the host antiviral responses to limit virus replication and production. The immune response is modulated, with the innate immunity as the first line defence before the adaptive immune system is generated. Both the host and virus can manipulate apoptosis and innate immune mechanisms as a form of defence or evasion strategy. Activation of extracellular signal-regulated kinase (ERK) was detected in cells infected with SARS-coronavirus and MERS-coronavirus and potentially associated with the facilitation of the ACE2 entry by the virus. Lim et al., showed that binding of SARS-coronavirus S protein to ACE2 receptor mediates ERK/MAPK activation and stimulates the upregulation of CCL2 chemokine, which is believed to be involved in respiratory inflammatory symptoms in SARS-coronavirus patients. The introduction of ERK pathway inhibitor was shown to inhibit MERS-coronavirus by approximatively 50%. Chloroquine, an antiviral used for malaria, was shown to phosphorylate MAPK pathway and, more recently, showed promising effects as COVID-19 treatment (Devaux C. A, et al., International Journal of Antimicrobial Agents, https://doi. org/10.1016/j.ijantimicag.2020.105938, 2020).

NF-κB was shown to control a broad range of biological processes, such as cell death, inflammation, innate and adaptive immune responses. NF-κB pathway has been shown to play an important role in coronavirus infections. In a preclinical model of SARS-coronavirus infection, treatment of infected lung cells with NF-κB inhibitors did not affect virus titres but reduced expression of TNF, CCL2 and CXCL2, suggesting that NF-κB is essential for SARS-coronavirus -mediated induction of pro-inflammatory cytokines. Interestingly, viruses can also use activation of MAPK and NF-κB pathways as strategies to subvert apoptosis. (Lim, Y. X. et al., Diseases, https://doi.org/10.3390/diseases4030026, 2016)

### Fenretinide's Lipid Modulation and its Pro-Resolving Effects on Inflammation.

In the context of Cystic Fibrosis (CF), fenretinide is being studied as a pro-resolving drug for inflammation. CF is characterized by an abnormally activated inflammatory response in the lung, which overreacts in the presence of pathogens and leads to irreversible lung damage. Further, fenretinide was shown to be a master regulator of key membrane lipids playing a dual role in both the resolution of inflammation, and the stabilization of Cystic Fibrosis Transmembrane Conductance Regulator (CFTR) in the epithelial apical membrane during inflammatory stress.

CFTR is an ion channel that mediates cAMP-stimulated chloride and bicarbonate secretion in the airways. Mutations in the CFTR gene cause defective CFTR ion channel function, resulting in disruption of chloride and sodium transport leading to viscous secretions in different exocrine tissues, with the most debilitating consequence being the mucus plug blocking the airways and impairing mucociliary clearance. Mutant CFTR also excite the immune-inflammatory response, resulting in exaggerated inflammatory response that is inefficient to eradicate pathogens, leading in persistent and unresolved inflammation, lung tissue destruction and scarring (Sly, P. D., et al., The New England Journal of Medicine, https://doi.org/10.1056/NEJMoa1301725, 2013). The aberrant inflammatory response in CF remains largely unaddressed, with high need for specific therapies capable of dampening the inflammation without interfering with its immune role in defending against infections (Harris, J. K. et al., Annals of the American Thoracic Society, https://doi.org/10.1513/AnnalsATS.201907-4930C, 2020).

Airway surface fluid from CF patients contains large concentrations of pro-inflammatory mediators including the tissue necrosis factor alpha (TNF-α), IL-1β, IL-6, IL-8, IL-17, and GM-CSF (Bonfield T L et al, Journal of Allergy and Clinical Immunology, https://doi.org/10.1016/S0091-6749(99)70116-8, 1999). The synthesis of these mediators is promoted by a few transcription factors including AP-1, nuclear factor (NF)-κB, and mitogen-activated protein kinase MAPK extracellular signal-regulated kinase (ERK 1/2). In addition to a heightened pro-inflammatory response, there appears to be inappropriately decreased counter-regulatory pathways, particularly those involving IL-10 and nitric oxide. Another mechanism inhibiting NF-kB activity occurs via up-regulation of peroxisome proliferator activating receptor (PPARγ). CF tissues appear to be deficient in PPARγ (Gautier E L et al, Nature Immunology, https://doi.org/10.1038/ni.2419, 2012) leading to an imbalance between inhibitors of kappa B (IKB) and NF-κB; and favors increased inflammation.

Due to the complex and paradoxical nature of the immune-inflammatory response in CF lung, the traditional anti-inflammatory or immunomodulation approaches have not resulted in a meaningful clinical outcome. The issue may reside in defective metabolism of Arachidonic Acid (AA) and docosahexaenoic acid (DHA), an emerging target supported by a strong rationale linked to the expression of CFTR (Torphy T. J. et al, Annals of the American Thoracic Society, https://doi.org/10.1513/AnnalsATS.201506-3610T, 2015). AA and DHA are two essential fatty acids playing a crucial role in maintaining an effective immune-inflammatory response. The CFTR gene defect causes exaggerated AA-mediated inflammation and reduced inflammation resolution due to low DHA levels, leading to persistent inflammatory response to lung infections. The abnormal fatty acids metabolism observed in CF patients has major impact on the cellular and intracellular phospholipid membranes. They are important regulators of signaling channels, protein function, permeability, caveolae building and are involved in the regulation of several genes expression (Strandvik B, Prostaglandins Leukotrienes and Essential Fatty Acids, https://doi.org/10.1016/j.plefa.2010.07.002, 2010). Lipid imbalance can be observed even in newborn mice with ablated CFTR gene, which are kept in pathogen free conditions (Guilbault C et al, American Journal of Respiratory Cell and Molecular Biology, https://doi.org/10.1165/rcmb.2006-0184TR, 2007). Furthermore, a correlation was shown between the severity of CF lung disease and lipid deregulation (Zhou J J et al, Journal of Membrane Biology, https://doi.org/10.1007/s00232-007-9056-6, 2007). Interestingly, the CF lipid imbalance “signature” does not appear to be related to the type of mutation.

Fenretinide pro-resolving effect on inflammation in CF is believed to be principally due to its ability to correct the defective lipid metabolism of key fatty acids involved in the resolution phase of inflammation. As opposed to a typical anti-inflammatory therapeutic effect that inhibits the onset mechanisms of the inflammation process, a pro-resolving therapeutic effect results from triggering body's own anti-inflammatory mechanism to reduce or stop the inflammation process. A correct balance between the onset phase and the resolution phase of the inflammation is crucial for an effective inflammatory response that plays its immune role, after which it resolves naturally to allow healing and preserve tissue homeostasis. The onset of inflammation is modulated by Arachidonic Acid (AA) pathway, and the resolution phase of inflammation principally involves Docosahexaenoic Acid (DHA) (Fullerton J. N. et al, Nature Reviews, Drug Discovery, https://https://doi.org/10.1038/nrd.2016.39, 2016). Exaggerated AA-mediated inflammation and inadequate inflammation resolution response due to downregulated DHA pathway, is one of the hallmarks of CF and is believed to chronic infection and lung destruction over time (Seegmiller A. C. et al, International Journal of Molecular Sciences, https://doi.org/10.3390/ijms150916083, 2014).

Fenretinide addresses the complex links between DHA metabolism and pro-resolving inflammatory signaling in the CF lung and modulates inflammation via a multi-target mechanism involving the pro-resolving modulation of ERK ((Lachance C. et al, Plos One, https://doi.org/10.1371/journal.pone.0074875, 2013), NF-κB (Vilela R. M., Science Direct, https://doi.org/10.1371/journal.pone.0074875, 2006) and PPARγ pathways. (Mcilroy G D et al, Diabetes, https://doi.org/10.2337/db12-04582013). All three targets, ERK 1/2, NF-κB and PPARγ are postulated to be important components of the endogenous resolution of inflammation and are all modulated by fenretinide. The timely resolution of inflammation is as important as the initiation phase and a good balance between pro-inflammatory and anti-inflammatory (pro-resolving) mediators is key to maintaining an efficient and harmless inflammatory response. (Kohli P et al, British Journal of Pharmacology, https://doi.org/10.1111/j.1476-5381.2009.00290.x, 2009). Incomplete resolution leads to chronic inflammation and destruction of lung tissue, and ultimately to lung insufficiency and impairment. More recently, it was demonstrated that fenretinide has the ability to inhibits the activity of cytosolic phospholipases (cPLA2), which was previously described as a factor for the abnormal high levels of AA in the cell membrane of CF patients. (Garic D. et al., BBA—Molecular and Cell Biology of Lipids, https://doi.org/10.1016/j.bbalip.2019.158538, 2019).

Fenretinide's effect on lipid metabolism and consequent modulation of inflammation resolution response was demonstrated in various animal models of inflammation and infection. Fenretinide was shown to correct the levels of DHA and AA essential fatty acids and sphingolipids imbalance in the lungs and plasma of a Cftr.KO mice model, resulting in reduction of lung inflammation and significant decrease in the pulmonary load of Pseudomonas aeruginosa (Guilbault C et al., American Journal of Respiratory Cell and Molecular Biology, https://doi.org/10.1165/rcmb.2008-02790C, 2009). Treatment of allergen-sensitized mice with fenretinide prevented induced changes in the AA and DHA levels, translating into a complete block of infiltration of inflammatory cells to the airways and dramatically diminished goblet cells proliferation (Kanagaratham C et al., American Journal of Respiratory Cell and Molecular Biology, https://doi.org/10.1165/rcmb.2014-01210C, 2014). Oral administration of fenretinide in a mice model of Spinal Cord Injury (SCI) produced a significant decrease in AA and increase in DHA in plasma and injured spinal cord tissue, leading to 1-reduced expression of pro-inflammatory genes and oxidative stress markers after SCI, 2-reduction of reactive microglia, 3-reduced tissue damage in the spinal cord and 4-improved locomotor recovery (Lopez-Vales R et al, The Journal of Neuroscience, https://doi.org/10.1523/JNEUROSCI.5770-09.2010, 2010).

Fenretinide has a lipid modulating effect on a mouse model of septic shock created induced by infection with Streptococcus suis (S. suis), an important swine pathogen, which was shown to lead to severe and frequently lethal meningitis in pork-industry workers in China that get infected with this bacterium. The cytokines storm caused by S. suis is responsible for early high mortality in septic shock-like syndrome cases. The study showed that mouse infection by S. suis was accompanied by an increase of AA and by a decrease of DHA. Treatment of mice with fenretinide significantly improved their survival by reducing systemic proinflammatory cytokines during the acute phase of an S. suis infection. These findings indicated a beneficial effect of fenretinide in diminishing the expression of inflammation and improving survival during an acute infection by a virulent S. suis strain. (Lachance, C. et al., Infection and Immunity, https://doi.org/10.1128/IAI.01524-132014) Macrophages infected with S. suis showed activation of ERK/MAPKs and cyclooxygenase-2 (COX2) upregulation. MAPKs play an important role in macrophage activation and the release of proinflammatory mediators. In the study, macrophages pretreated with fenretinide prior to S. suis infection showed a significant reduction in ERK1/2 activation compared to nontreated S. suis-infected macrophages.

LAU-7b pro-resolving effect was investigated in a Phase 1 b dose-ascending, placebo-controlled trial in adult CF patients. In a subgroup of patients experiencing pulmonary exacerbation (PEx), fenretinide normalized the lipidomic markers in a dose-response manner and the profile of key lipidomic markers (DHA, AA) in these patients was shown to be superior at the onset of PEx to values measured in a similar population in a natural history study where exacerbating patients were treated with the standard of care for exacerbation. Furthermore, treatment with fenretinide also appeared to improve the plasma levels of IL-6, IL-8, IL-10 and neutrophils count at the onset of the PEx episode. A better systemic anti-inflammatory profile at onset of PEx was recently shown to correlate with increased odds to better respond to antibiotics for PEx (Sagel S. D. et al, ATS Journals, https://doi.org/10.1513/AnnalsATS.201410-4930C, 2015). Overall, these data demonstrate that a normalized lipidomic profile in patients experiencing an exaggerated inflammatory response is important to keep a balanced cytokine level, and potentially protective for the lungs of the patients during exacerbation episodes. (Radzioch D. et al, ATS Journals, https://www.atsjournals.org/doi/abs/10.1164/ajrccm-conference.2016.193.1, 2016).

### Fenretinide Effect on Membrane Lipids Composition and Ion Channels Activity.

More recent data shows fenretinide's potential to act on membrane sphingolipid self-protection mechanism (Garic et al. Journal of Molecular Medicine, (https://doi.org/10.1007/s00109-017-1564-y, 2017) and to have effects on CFTR protein insertion and stability in the airway epithelial apical membrane, an effect synergistically enhanced in the presence of a CFTR corrector (Abu-Arish, A. et. al., Journal of General Physiology, http://doi.org/10.1085/jgp.201812143, 2019), thus confirming the important link between inflammation and the basic defect in CF. Ceramide-rich platforms are particularly interesting in the context of CF because ceramides and other lipids are altered in CF cells. Very long chain ceramide (VLCC) such as C24:0 are considered anti-inflammatory are decreased in CF patients and CFTR knock-out mice ((Guilbault C et al, American Journal of Respiratory Cell and Molecular Biology, https://doi.org/10.1165/rcmb.2008-02790C, 2009)), while proinflammatory long chain ceramides (LCCs; e.g., C16:0) are increased (Teichgraber, V. et al., Nature Medicine, https://doi.org/10.1038/nm1748, 2008)

Fenretinide corrected the imbalance between VLCCs and LCCs in CFTR-null mice (Guilbault C et al, American Journal of Respiratory Cell and Molecular Biology, https://doi.org/10.1165/rcmb.2008-02790C, 2009). Fenretinide was shown to down-regulate expression of the endoplasmic reticulum enzyme Cers5, which increases synthesis of VLCCs by Cers2:Cers5 heterodimers relative to synthesis of LCCs by Cers5:Cers5 homodimers, thereby correcting the ceramide imbalance (Garic, D. et al. Journal of Molecular Medicine, https://doi.org/10.1007/s00109-017-1564-y, 2017).

### Effect of Fenretinide on SARS-coronavirus Infection.

Recent data from an in vitro high-content screening (HCS) strategy for repurposing newly identified inhibitors of MERS-CoV, in an effort to identify potential therapeutic options for COVID-19, showed that fenretinide was able to inhibit a MERS-CoV clinical isolate with an 50% inhibitory concentration (IC50) at a concentration of 2.8 μM. The test evaluated anti-MERS-CoV activity by determining the levels of the viral spike (S) protein expression of infected Vero cells by immunofluorescence analysis (IFA). (Meehyun, K., et al., BioRxiv, https://doi.org/10.1101/2020.02.25.965582, 2020).

Broader antiviral effects of fenretinide were also demonstrated in the past, at low concentrations. Indeed, fenretinide was shown to have potent activity against Zika virus in vitro by targeting nonstructural protein 5 (NSPS) (Chunxiao Wang, Biochemical and Biophysical Research Communications, https://doi.org/10.1016/j.bbrc.2017.10.016, 2017). Previously, fenretinide also showed potent antiviral activity against Dengue fever disease, by targeting NSPS and also inducing phosphorylation of eukaryotic translation initiation factor 2α (eIF2α).

Intriguingly, the authors found that fenretinide leads to specific activation of the unfolded protein response (UPR), culminating in rapid elimination of viral RNA from the infected cells. They also showed that fenretinide can protect against Dengue infection in a lethal mouse model. Since Dengue disease pathology is in part due to an overactive inflammatory response, the authors discussed the possibility that fenretinide modulation of the UPR may lead to a rebalancing of cytokine levels to promote viral clearance. Consistent with this, cytokine levels in fenretinide—treated mice are decreased overall relative to the infection control group. (Johanna E. Fraser, The Journal of Infectious Diseases, https://doi.org/10.1093/infdis/jiu319, 2014).

The mechanisms and results disclosed herein demonstrate the novel discovery that fenretinide is effective in treating SARS-coronavirus infection, SARS-coronavirus ARDS, pneumonia induced ARDS, and certain conditions associated with or arising from SARS-coronavirus such as ARDS related hypoxia.

Acute respiratory distress syndrome (ARDS) is characterized by lung inflammation and pulmonary edema, leading to arterial hypoxemia and death if the hypoxemia is severe. Strategies to correct hypoxemia have the potential to improve clinical outcomes in ARDS. As demonstrated herein, administration of formulations and dosages of fenretinide in accordance with the present invention, can prevent the hypoxemia induced by ARDS, as measured by the arterial blood oxygen saturation (SpO₂).

The present invention provides for the novel and unexpected benefit of fenretinide administration, in accordance with the present invention, as a means to reduce or prevent the decrease of circulating reticulocytes in the blood caused by inflammation, and to maintain blood circulating reticulocytes at those levels present in the absence of inflammation. Reticulocytes are immature red blood cells that are developed in the bone marrow as part of the process of erythropoiesis and are often produced as a compensatory mechanism against anemia of inflammation during chronic infection, ARDS, or sepsis. Anemia is a condition characterized by reduction of the circulatory red blood cells necessary to provide adequate tissue oxygenation and is commonly associated with critical illness such as ARDS and sepsis. Anemia is also described as a factor contributing to poor outcomes observed in patients suffering from SARS-coronavirus infection. The unexpected impact of fenretinide administration on maintaining or increasing the circulating blood reticulocytes in animal models of acute lung injury are consistent with protection or stimulation of the erythropoiesis process in ARDS. Although the present invention is not bound or limited by any one mechanism of action, this provides further support for the observed beneficial impact of fenretinide administration on blood oxygen saturation and for use of fenretinide to prevent and/or treat hypoxemia; all in accordance with the present invention.

### EXAMPLE 1

### Correlation of Oral LAU-7b to Plasma Fenretinide Concentration.

25 mg/kg, 62.5 mg/kg, and 125 mg/kg LAU-7b SDI (an improved oral formulation of fenretinide, formulated as spray dried solid amorphous dispersion, with each 2.5mg of LAU-7b or LAU-7b-SDI containing 1 mg fenretinide, 1.49 mg povidone, 0.006 mg beta-hydroxy acid, and 0.004 mg butylated hydroxytoluene) was orally administered by gavage to male C57B:/6 mice obtained from Charles River Laboratories, which correlated to an oral administration of 10 mg/kg, 25 mg/kg and 50 mg/kg fenretinide. Mean concentration of plasma fenretinide levels in the blood was determined 2 hours following the oral administration; and mean fenretinide concentration of 3.3 μM, 7.2 μM, and 8.6 μM was obtained for the oral LAU-7b SDI administrations of 25 mg/kg, 62.5 mg/kg, and 125 mg/kg respectively.

### EXAMPLE 2

### Viral Inhibition of SARS-CoV-2 Coronavirus by Fenretinide

Vero E6 cells were grown to a confluency of between 80%-100% in 24 well plates; and 0.2 mL of suspension of SARS-CoV-2 coronavirus in Modified Eagles Medium (MEM) with 2% fetal bovine serum added to the wells and incubated at 37° C. for 90 minutes, to allow viral adsorption. The MEM suspension was removed, and an overlay of agarose and fenretinide, agarose and remdesivir, or agarose fenretinide and remdesivir was added; all in MEM with 2% fetal bovine serum. Seven different concentrations of fenretinide and remdesivir were tested, in triplicate; with the Vero E6 cells incubated for 3 to 4 days at 37° C.

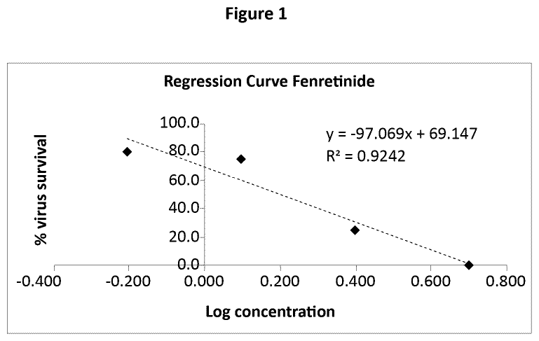

Following incubation, the cells were fixed with 0.5mL of 3.7% formaldehyde for 30-60 minutes; following which the agarose was removed and the cells stained with 0.8% crystal violet in ethanol. The number of viral plaques in each well was determined using an inverted microscope, and the concentration of fenretinide, remdesivir, or fenretinide and remdesivir needed to reduce the number of plaques by 50% (IC50). Remdesivir, an adenosine nucleoside analogue with known antiviral properties was used as a well-established positive control. Log values of tested fenretinide concentrations and corresponding percentage values of virus survival were plotted in a XY plot to calculate 50% inhibition of virus survival (IC₅₀) by linear regression analysis (FIG. 1). Remdesivir, an exemplary antiviral from the broader class of delayed chain terminators (positive control) also reduced progressively the number of plaques in the Vero 6 seeded wells infected by SARS-Cov-2 virus from 90.9% at 0.625 μM to 0% at 10 μM concentration. No cytopathic effect of remdesivir was observed up to 40 μM concentrations. Mean value of formed plaques in untreated wells was considered as 100% virus survival. Fenretinide treatment inhibited the virus survival in the Vero 6 cells with the IC50 of 1.57 μM (R2=0.92).

Table 1 presents plaque forming unit counts (individual counts/well and mean values, n=3) and mean virus survival (%) at fenretinide concentrations, obtained by serial, two-fold dilutions. Table 2 presents plaque forming unit counts (individual counts/well and mean values, n=3) and mean virus survival (%) at remdesivir concentrations, obtained by serial, two-fold dilutions. Table 3 presents plaque forming unit counts (individual counts/well and mean values, n=3) and mean virus survival (%) following combination treatment of fenretinide with remdesivir at concentrations obtained by serial, two-fold dilutions. Clear demonstration of antiviral effects of fenretinide, comparable to the known antiviral remdesivir, was obtained.

TABLE 1
Plaque forming unit counts of SARS-CoV-2 coronavirus
infected Vero E6 cells treated with fenretinide.

| SARS-CoV-2 | | | | | | |
|---|---|---|---|---|---|---|
| Conc | | Fenretinide | | | | |
| Fenretinide | Log | Wells | | | Mean | |
| (μM) | conc | 1 | 2 | 3 | PFU/well | % survival |
| 0 | | 24 | 23 | 30 | 25.7 | 100.0 |
| 0.625 | −0.204 | 21 | 19 | 22 | 20.7 | 80.5 |
| 1.25 | 0.097 | 14 | 31 | 13 | 19.3 | 75.3 |
| 2.5 | 0.398 | 6 | 3 | 10 | 6.3 | 24.7 |
| 5 | 0.699 | 0 | 0 | 0 | 0.0 | 0.0 |
| 10 | 1.000 | NA | NA | NA | | |
| 20 | 1.301 | NA | NA | NA | | |

TABLE 2
Plaque forming unit counts of SARS-CoV-2 coronavirus
infected Vero E6 cells treated with remdesivir.

| SARS-CoV-2 | | | | | | |
|---|---|---|---|---|---|---|
| Conc | | Remdesivir | | | | |
| Remdesivir | Log | Wells | | | Mean | % virus |
| (μM) | conc | 1 | 2 | 3 | PFU/well | survival |
| 0 | | 30 | 26 | 32 | 29.3 | 100.0 |
| 0.625 | −0.204 | 25 | 25 | 20 | 23.3 | 90.9 |
| 1.25 | 0.097 | 10 | 11 | 12 | 11.0 | 42.9 |
| 2.5 | 0.398 | 5 | 5 | 2 | 4.0 | 15.6 |
| 5 | 0.699 | 1 | 1 | 0 | 0.7 | 2.6 |
| 10 | 1.000 | 0 | 0 | 0 | 0.0 | 0.0 |
| 20 | 1.301 | 0 | 0 | 0 | 0.0 | 0.0 |
| 40 | 1.602 | 0 | 0 | 0 | 0.0 | 0.0 |

TABLE 3
Plaque forming unit counts of SARS-CoV-2 coronavirus infected
Vero E6 cells treated with remdesivir and fenretinide.

| SARS-CoV-2 | | | | | |
|---|---|---|---|---|---|
| Concentration | Remdesivir-Fenretinide | | | | |
| remdesivir:fenretinide | Wells | | | Mean | % virus |
| (μM) | 1 | 2 | 3 | PFU/well | survival |
| 0 | 31 | 21 | 35 | 29.0 | 100.0 |
| 0.09:0.078 | 29 | 28 | 25 | 27.3 | 94.3 |
| 0.1875:0.15625 | 18 | 20 | 24 | 20.7 | 71.3 |
| 0.375:0.3125 | 11 | 15 | 21 | 15.7 | 54.0 |
| 0.75:0.625 | 18 | 16 | 26 | 20.0 | 69.0 |
| 1.5:1.25 | 9 | 8 | 15 | 10.7 | 36.8 |
| 3:2.5 | 0 | 0 | 0 | 0.0 | 0.0 |
| 6:5 | 0 | 0 | 0 | 0.0 | 0.0 |

The obtained data for fenretinide and remdesivir concentration was entered in the Compusyn software, version 1.0 (ComboSyn, Inc., Paramus, N.J.) and the synergism, additivity or antagonism of the two drugs were calculated using the combination index (CI) values. A weighted average CI (CW_{wt}) was calculated for each combination as (CI₅₀+2xCI₇₅+3xCI₉₀+4xCI₉₅)/10 to estimate drug combination effects at high levels of virus inhibition and to increase therapeutic relevance. Drug combination effects were defined as CI_{wt}<0.7, synergism; CI_{wt}>0.7 and <0.9, moderate synergism; CI_{wt}>0.9 and <1.2, additivity; CI_{wt}>1.2 and <1.45, moderate antagonism and CI_{wt}>1.45, antagonism (Chou, T. C et al. Pharmacology Reviews, http://doi.org/10.1124/pr.58.3.10 58(3):621-81, 2016; Drouot, E. et al., Antiviral Therapy 21(6):535-539, http://doi.org/10.3851/IMP3028, 2016). Based upon calculated CI₅₀ of 1.253, CI₇₅ of 0.677, CI₉₀ of 0.368 and CI₉₅ of 0.245; a CI_{wt} of 0.47 was obtained, indicating a synergism between the antiviral effects of fenretinide and remdesivir; and by extension an anticipated synergism between the antiviral effects of fenretinide and antivirals generally known in the art as delayed chain terminators, including but not limited to penciclovir, cidofivir, entecavir and remdeivir.

### EXAMPLE 2

### Therapeutic Effects of LAU-7b LPS Induced ARDS Mouse Model (Tracheal Instillation of 50 μg of LPS).

To demonstrate the efficacy of fenretinide in reducing or ameliorating ARDS in mice, an LPS installation mouse model was used to simulate ARDS. The LPS-induced model of ARDS is a well-established model of lung injury that replicates most of the lung complications of human COVID-19. Although the current animal models of SARS-coronavirus infection are able to reproduce the viral infection in upper and lower respiratory tract and some of the lung pathology, these lung complications are mild and the animals are able to recover without developing a severe manifestations such as ARDS or the cytokine storm observed in humans, indicating that a wide gap separates the animal models from the full spectrum of COVID-19 in humans (Ehaideb, S. et al. Critical Care 24:594 https://doi.org/10.1186/s13054-020-03304-8, 2020). These fundamental differences are less of a problem for the exploration of virus-directed antivirals or other early therapies such as therapeutic antibodies, but are a real challenge when the investigation therapeutic is directed at the host response, complications of the disease, and prevention of the ARDS.

Male C57BL/6 mice from Charles River Laboratories, weighing 20 grams to 25 grams, were administered with a single intratracheal instillation of 50 μg of LPS dissolved in sterile 0.9% saline (Groups 2 and 3) or 50 μg of 0.9% saline (Group 1). Two hours after LPS instillation, animals from Group 3 were administered 25 mg/kg of LAU-7b SDI by oral gavage in a total volume of 10mL/kg; and Group 1 and Group 2 received vehicle only at a volume of 10 mL/kg. At 24 hours after arterial oxygen saturation (SpO₂), heart rate, respiratory parameters (whole-body plethysmography) were recorded; and blood was collected for determination of red blood cell count, hematocrit, mean corpuscular volume (MCV), and white blood cells total and differential counts. Plasma was retained for quantification of the chemokine and cytokine levels in plasma. The animals were sacrificed, and the thoracic cavity opened to expose the lungs and trachea, which were connected to the cannula of a perfusion system and 0.9 mL of cold Phosphate Buffered Saline with 900 μL of 1X solution of Protease Inhibitor injected into the trachea and perfused through the lungs; thereby generating bronchoalveolar lavage fluid (BALF) which was maintained for further analysis. BALF provides insight to the cellular, cytokine and chemokine environment of the lungs, as opposed to the systemic values obtained from blood analysis.

Three additional sets of mice, Groups 4, 5 and 6 were established, and Groups 5 and 6 received a single instillation of 50 μg of LPS dissolved in sterile 0.9% saline; and two hours after LPS instillation mice in Group 6 received a dose of 25 mg/kg of LAU-7b SDI by oral gavage in a total volume of 10 mL/kg, and again at 24 hours and 48 hours. Group 4 and 5 received vehicle only at a volume of 10 mL/kg. At 72 hours the animals in Group 4, 5 and 6 were sacrificed and samples obtained, both systemic and BALF, as with the animals described in the preceding 24-hour assessment.

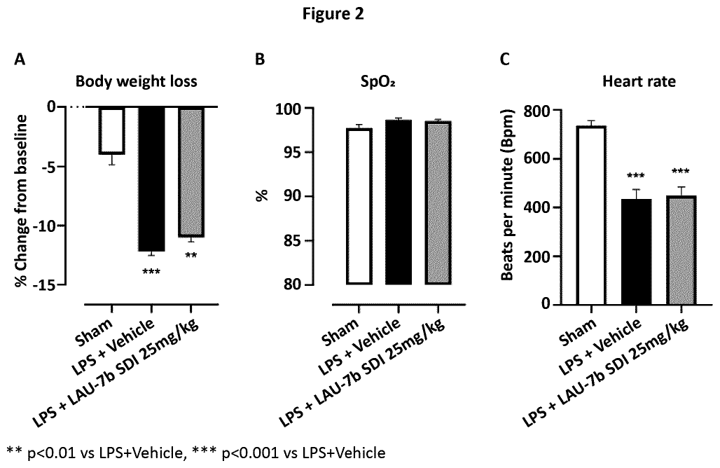

As shown in FIG. 2, several physiological parameters for Groups 1 (Sham) Group 2 (LPS) and Group 3 (LAU-7b SDI) observed to be affected by the LPS dose.

Significant body weight loss (A) and heart rate reduction (C) occurred at 24 hours in Vehicle and LAU-7b SDI groups. SpO₂ was slightly increased in both LPS groups compared to Sham mice (B). No statistical difference was observed between the Vehicle and LAU-7b SDI groups for any parameter, however body weight loss was less pronounced in LAU-7b SDI treated mice.

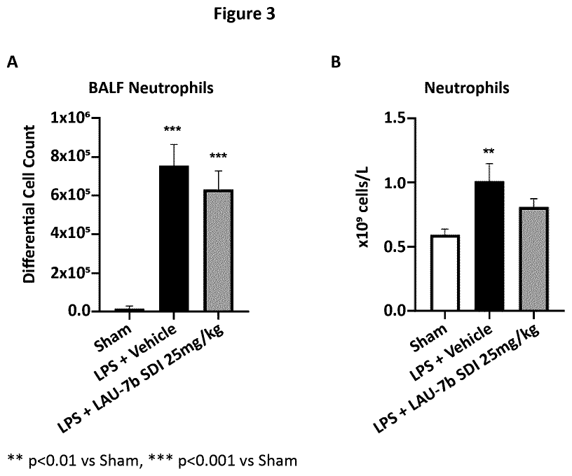

FIG. 3. shows BALF neutrophil cell counts for Groups 1 (Sham), Group 2 (LPS), and Group 3 (LAU-7b SDI), and as compared to LPS mice, LAU-7b SDI had lower neutrophil cell count (A). FIG. 3 also shows total and differential blood neutrophil cell counts for Groups 1 (Sham) Group 2 (LPS) and Group 3 (LAU-7b SDI); with LPS mice having higher neutrophil counts as compared to the LAU-7b SDI group (B).

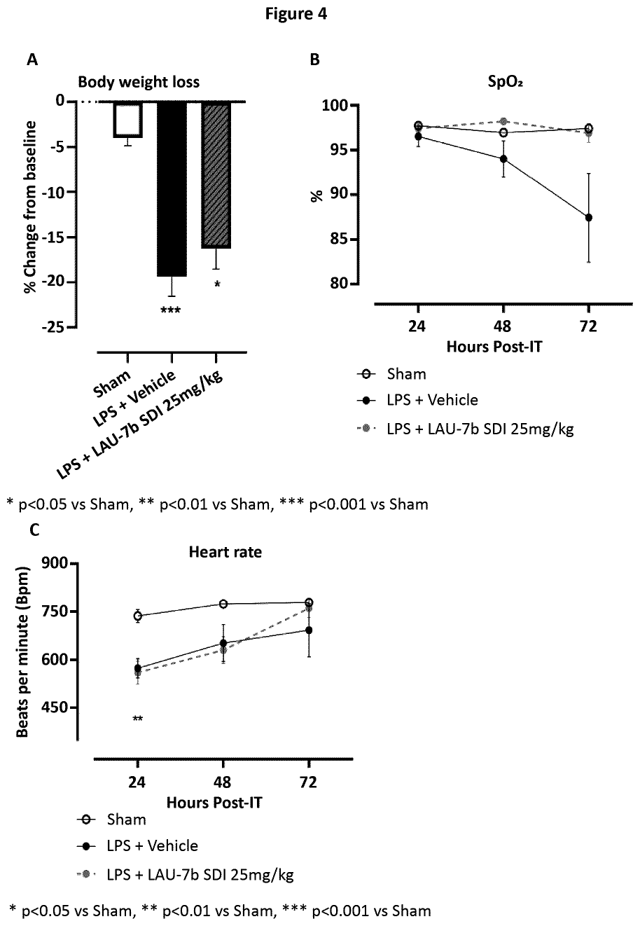

As shown in FIG. 4 both groups that received LPS, showed statistically significant body weight loss (close to 20%) at 72 h post LPS administration as compared to Sham (A). A milder reduction of the body weight loss, compared to the Sham mice, was observed in the group treated with 25 mg/kg of LAU-7b SDI. LPS mice showed continuous reduction of 402, during the study reaching saturation below 90% at 72 hours (B). The treatment with LAU-7b SDI at 25 mg/kg dose completely prevented reduction of blood oxygen saturation at 48 and 72 h, this effect, however, was not statistically significant. Statistically significant reduction in heart rate observed at 24 h in both LPS and LAU-7b SDI improved at 48 h and reached the Sham values at 72 h in the group tested with LAU-7b SDI (C). The improvement of heart rate at 72 h was less pronounced in the LPS group, but not statistically different for the LAU-7b SDI treated group.

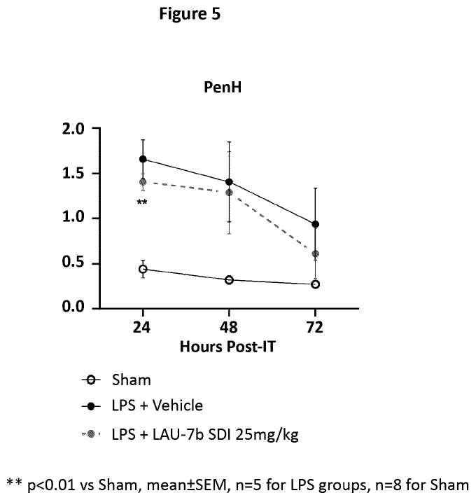

FIG. 5 presents calculated pulmonary congestion index values (PenH) values as a measure of respiratory parameters for Group 4 (Sham), Group 5 (LPS), and Group 6 (LAU-7b SDI). Compared to the Sham mice, both LPS and LAU-7b SDI had significantly higher PenH values at 24 h. With the time the PenH values decreased but did not completely recover to the Sham value at 72 h post LPS instillation. Although, no statistical difference between the LPS and LAU-7b SDI groups was observed at any timepoint, LAU-7b SDI treatment provided respiration protection, particularly at 72 h post-LPS.

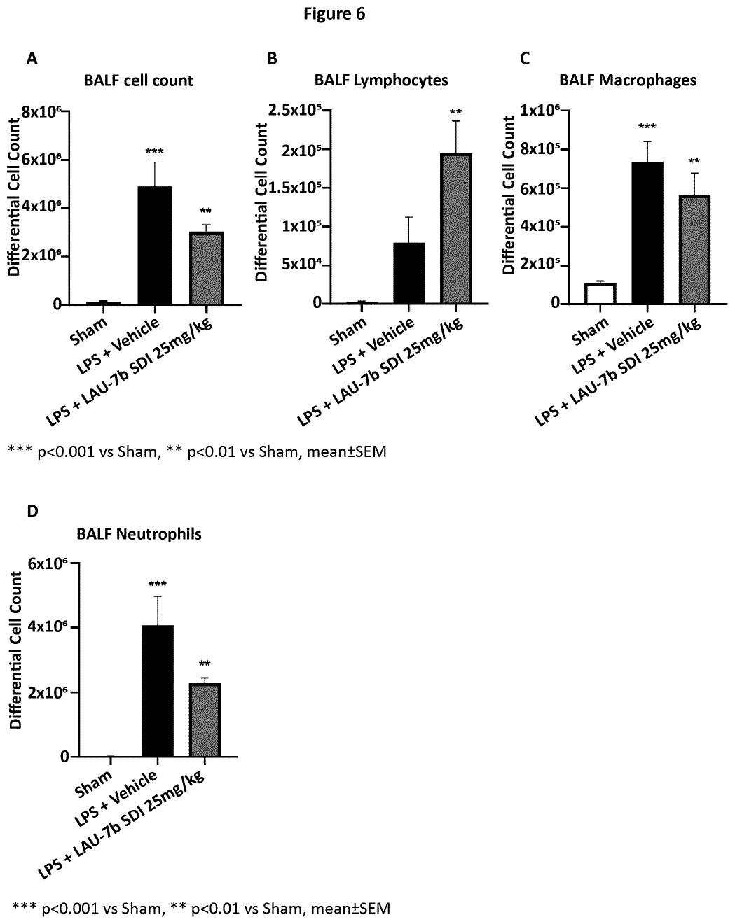

FIG. 6 presents the total and differential cell counts in BALF collected at the sacrifice 72 hours for Group 4 (Sham), Group 5 (LPS), and Group 6 (LAU-7b SDI). Compared to the Sham group, the LPS group showed significantly higher BALF total cell (A), macrophage (C), and neutrophils cells (D) counts that were partially reduced in the LAU-7b SDI groups. Compared to the Sham mice, an increase in lymphocyte count occurred in both the LPS and LAU-7b SDI groups (B), however this increase was statistically significant only in LAU-7b SDI group.

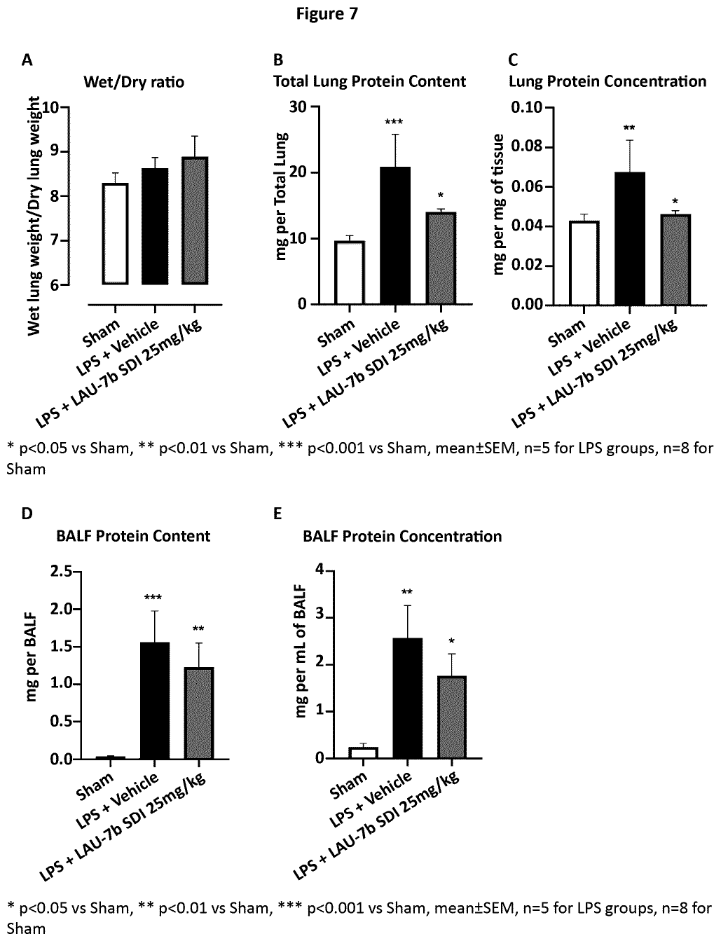

FIG. 7 presents the lung wet/dry ratio (A), lung protein content (B) and lung protein concentration (C) for Group 4 (Sham), Group 5 (LPS), and Group 6 (LAU-7b SDI). Compared to the Sham mice, both LPS groups had higher lung wet/dry ratio, however this difference was not statistically significant compared to the Sham group. Compared to the Sham mice, both LPS and LAU-7b SDI groups had significantly higher protein content and lung protein concentration at 72 hours post-LPS instillation. The LAU-7b SDI treatment showed a tendency to reduce the total lung protein content (B) and concentration (C), however this reduction was not statistically significant compared to the LPS group. Similar to the total lung protein content (B) the BALF protein content increased significantly following LPS instillation (D). Compared to the Sham group, the LPS and LAU-7b SDI groups had statistically significant higher BALF protein content (D) and concentration (E). Treatment with LAU-7b SDI showed a tendency to reduce the BALF protein content and concentration compared to the LPS.

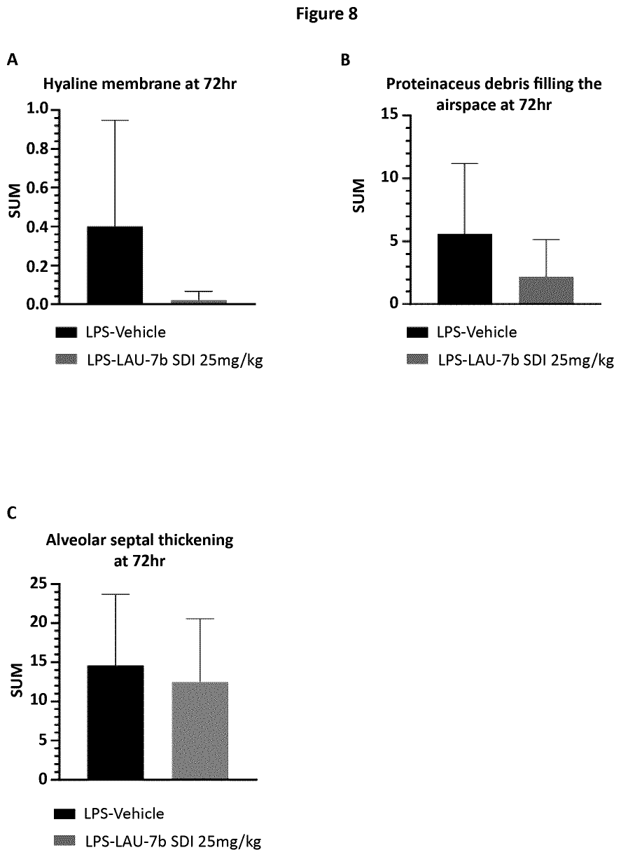

FIG. 8 presents detailed histopathology analysis of lung tissue in groups sacrificed at 72 h post-LPS, which showed a tendency to reduce the hyaline membranes and proteinaceous debris in the airspace as well as the alveolar septal thickening in the group treated with LAU-7b SDI.

LAU-7b SDI oral treatment at the dose of 25 mg/kg (10 mg/kg of fenretinide) led to a reduction of several proinflammatory cytokines in the BALF and plasma, as well as reduction of lung and BALF protein and neutrophil contents. Pulmonary inflammation in ARDS models is mediated by breaking the balance between proinflammatory and anti-inflammatory cytokines and chemokines. Those molecules can be measured in

BALF and plasma. Proinflammatory molecules such as IL-1, IL-6, IL-12, IL-17 and TNF-αare detrimental and key in the development of the disease in both humans as in animals (Matute-Bello et al. Am J Respir Cell Mol Biol.; 44(5):725-738. doi:10.1165/rcmb.2009-0210ST; 2011; McGonagle D, et al. Autoimmun Rev., 19(6):102537. doi:10.1016/j.autrev.2020.102537, 2020). Others showed the correlation of certain cytokines and COVID-19 with disease severity. Indeed, high plasma levels of IL-6 and TNF-α are an indicator of acute lung inflammation in COVID-19 infection, high plasma levels of IL-3 and IL-17 have been associated with viral load and severity, and IL-2 has been shown to play a key role in the proliferation of T-cells which are associated with immune defense pathogens (Costela-Ruiz V J, et al. Cytokine Growth Factor Revue; 54:62-75, doi: 10.1016/j.cytogfr.2020.06.0012020).

To parallel the clinical setting, those cytokines/chemokines were measured at 24 h in the plasma of LPS-induced ARDS animals treated or not with LAU-7b SDI , with LAU-7b SDI oral treatment at the dose of 25 mg/kg (containing 10 mg/kg of fenretinide) showing statistically significant reduction of plasmatic levels of IL-1 α, IL-3, TNF-α, as well as numeric reduction in the plasmatic levels of IL-6, IL-7, IL-17, and increase in the plasmatic levels of IL-2 and VEGF. The most important changes in cytokines and chemokines associated with LAU-7b SDI treatment at 72 h were the numeric reduction of IL-6, TNF-a and RANTES levels, both in plasma and BALF. The increase of vascular endothelial growth factor (VEGF) (plasma and BALF) may reflect the regeneration of injured lung blood vessels and repair of the alveolar-capillary membrane, and therefore playing an important role in the pathology of ARDS.

Treatment with LAU-7b SDI increased plasma and BALF VEGF levels at 72 h. The cytokine IL-3 is not involved in the cytokine storm; however, it was shown to be an independent prognostic marker for the outcome of COVID-19 patients. Low plasma IL-3 levels in severe COVID-19 patients presenting with ARDS are associated with increased disease severity, increased viral load and high mortality rates. Patients older than 65 years showed reduced plasma IL-3 levels compared with patients younger than 65. Therefore, IL-3 is an early predictive marker helping to identify patients at high risk (Benard A et al, Nat Commun 12, 1112.https://doi.org/10.1038/s41467-021-21310-4 , 2021). In particular plasmatic IL-3 was significantly reduced in the LAU-7b SDI group as compared to LPS group, after 24 hours. Increased IL-3 has been identified as correlative with the cytokine storm experienced by SARS-coronavirus patients and may represent a marker for identification of therapeutic efficacy of LAU-7b or fenretinide treatment in a patient experiencing ARDS. Further, although demonstrating trends for increased plasmatic IL-2 in LAU-7b SDI group, as compared to the LPS, group at 24 h and 72 h, the effect is supportive of a further protective or ameliorating effect of LAU-7b SDI in diminishing the ARDS related inflammation and cytokine storm.

### EXAMPLE 3

### Therapeutic Effects of Oral and Inhaled LAU-7b SDI in LPS Induced ARDS Mouse Model (Tracheal Instillation of 60 μg of LPS).

LAU-7b SDI at 25 mg/kg (10 mg/kg of fenretinide) was formulated in 0.5% methylcellulose and administered by oral gavage to C57BL/6 mice, providing a Cmax plasma concentration of 2-3 μM in the mice. An inhaled formulation of fenretinide was prepared and administered to C57BL/6 mice to provide an effective local fenretinide concentration in the lung of the mice of 1-3 μM, while limiting the system exposure of the mice to the drug. Two inhaled dosages of the fenretinide were tested, administered by way of nebulization of the fenretinide inhaled formulation containing 0.65 mg/ml fenretinide for 30 minutes or 60 minutes, resulting in lung delivery of an inhaled fenretinide dosage of 1.8 μg/kg and 3.6 μg/kg, respectively.

The inhaled fenretinide dosage was prepared as follows. Fenretinide stock solution was prepared in 100% DMSO at 65 mg/mL. A selected volume of the stock solution was gradually diluted 100× in PBS 1X+0.1% Tween-80 solution to obtain the final fenretinide concentration of 0.65 mg/mL that was used for the nebulization. The final solution of 0.65 mg/mL fenretinide contained 1% DMSO. The control LPS mice received the vehicle only, containing PBS 1X+0.1% Tween-80 and 1% DMSO. An Aerogen nebulizer was used for lung delivery of the final formulation of fenretinide containing 0.65 mg/mL of fenretinide connected to an aerosol system (Oro-Nasal and Respiratory Exposure System, CH Technologies, Westwood, N.J.) operating at a flow rate of 6 L/min. Duration of the nebulization was 30 min/mouse for the low dose 1.8 μg/kg and 60 min/mouse for the dose of 3.6 μg/kg. The calculated effective fenretinide dose delivered to the lungs was between 1-3 μM. These concentrations were confirmed by analysis of the lung tissue of the exposed mice

To assess the therapeutic effect of fenretinide in a mouse model of acute lung injury, a single intratracheal instillation of 60 μg lipopolysaccharide (LPS) dose prepared as a solution of 60 μL of 1 mg/mL LPS in 0.9% saline. This is an increased dosage of LPS as compared to Example 2, intended to increase the lung damage experienced in the mouse model. Treatment of the mice, both oral and inhaled at the two inhaled dosages, was initiated two hours following LPS instillation; and the animals sacrificed 24 hours post LPS instillation, or 72 hours post LPS instillation. For those animals sacrificed 72 hours post LPS instillation, further oral or inhaled dosages of fenretinide were administered at 24 and 48 hours post LPS instillation. “Sham” mice received no LPS but respective vehicle while the negative control group of mice received LPS and vehicle but no fenretinide.

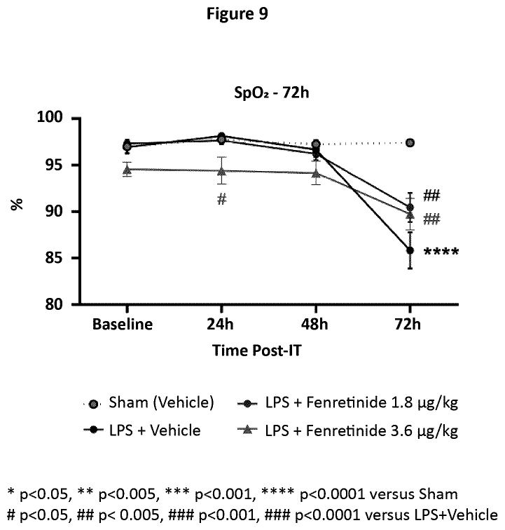

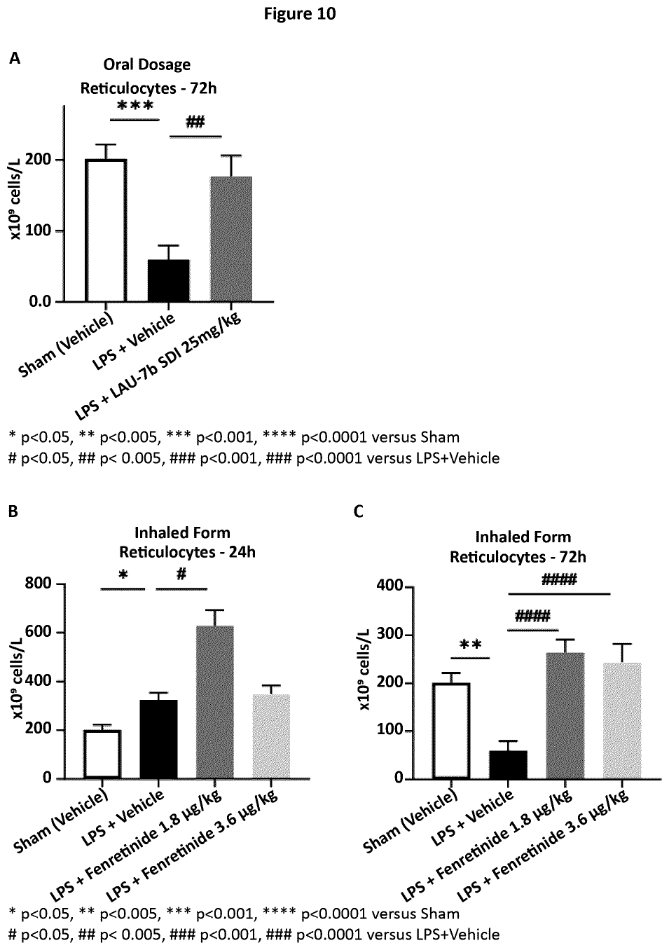

FIG. 9 presents the oxygen saturation (SpO₂) as measured in the blood of Sham, negative control and mice receiving 1.8 μg/kg and 3.6 μg/kg fenretinide in an inhaled form. Both dosages of inhaled fenretinide partially, but significantly, alleviated the reduction of blood oxygen saturation at 72 h. FIG. 10 presents the reticulocyte count in the blood of mice receiving either oral fenretinide as LAU-7b SDI at 72 hours (A), or each of the two inhaled dosages of fenretinide at 24 (B) and 72 hours (C). At 72 hours fenretinide administration, either oral or at either inhaled dosage of 1.8 μg/kg or 3.6 μg/kg fenretinide, significantly increased reticulocyte counts as compared to the LPS negative control group.

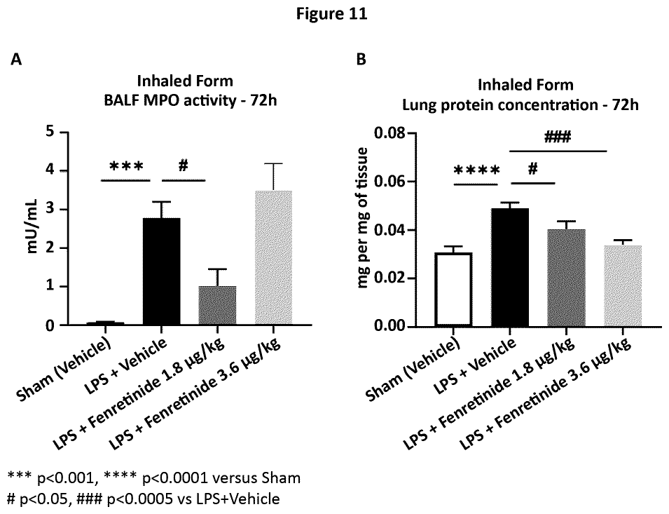

Myeloperoxidase (MPO) is a key element of the innate immune system and is released primarily by neutrophils to provide defence against invading pathogens. The myeloperoxidase (MPO) activity has been known as a biomarker to assess the infiltration of neutrophils and macrophages within pulmonary tissues, which is a hallmark of ARDS and COVID-19 lung complications (Goud P. T. et al, 2021, nt J Biol Sci. 2021; 17(1): 62-72. doi: 10.7150/ijbs.51811). As presented in FIG. 11, at 72 hours a dosage of 1.8 μg/kg, inhaled fenretinide reduced MPO activity in the LPS induced ARDS mouse model and was statistically significant as compared to the negative control (A); while both 1.8 μg/kg and 3.6 μg/kg of inhaled fenretinide reduced the lung protein concentration, as compared to Sham, at 72 hours.

### EXAMPLE 4

### Therapeutic Treatment of SARS-coronavirus Infection

Administration of an effective amount of a pharmaceutical composition comprising fenretinide (such as LAU-7b) is undertaken with a human patient experiencing a SARS-coronavirus infection and presenting symptoms; following which the subject subsequently exhibits improvements of clinical symptoms associated with SARS-coronavirus.

Administration is undertaken by providing the patient an oral formulation comprising a pharmaceutically acceptable salt of fenretinide or analogs thereof such as LAU-7b; and it is contemplated to optionally include a pharmaceutically acceptable excipient as part of the oral formulation. Within a period of time, up to about 21 days, symptoms associated with SARS-coronavirus infection are reduced.

Administration may include oral administration of LAU-7b to a human, in the form of three capsules containing 100 mg LAU-7b once per day for three days; followed by oral administration of two capsules containing 100 mg of LAU-7b once per day for 11 days.

### EXAMPLE 4

### Therapeutic Treatment of SARS-Coronavirus Associated Pneumonia

Administration of an effective amount of a pharmaceutical composition comprising fenretinide (such as LAU-7b) is undertaken with a human patient experiencing a SARS-coronavirus infection and presenting pneumonia symptoms; following which the subject subsequently exhibits improvements of clinical symptoms associated with pneumonia. It is contemplated that the pneumonia is caused by the SARS-coronavirus viral infection alone, a SARS-coronavirus complications with bacterial infection, or a combination of both.

Administration is undertaken by providing the patient an oral formulation comprising a pharmaceutically acceptable salt of fenretinide or analogs thereof such as LAU-7b; and it is contemplated to optionally include a pharmaceutically acceptable excipient as part of the oral formulation. Within a period of time, up to about 21 days, symptoms associated with pneumonia are reduced.

### EXAMPLE 5

### Therapeutic Treatment of SARS-Coronavirus ARDS

Administration of an effective amount of a pharmaceutical composition comprising fenretinide (such as LAU-7b) is undertaken with a human patient experiencing a

SARS-coronavirus infection and presenting ARDS symptoms; following which the subject subsequently exhibits improvements of clinical symptoms associated with ARDS. It is contemplated that the ARDS is caused by either pneumonia, the viral infection alone, or a combination of both.

Administration is undertaken by providing the patient an oral formulation comprising a pharmaceutically acceptable salt of fenretinide or analogs thereof such as LAU-7b; and it is contemplated to optionally include a pharmaceutically acceptable excipient as part of the oral formulation. Within a period of time, up to about 21 days, symptoms associated with ARDS are reduced.

### EXAMPLE 6

### Therapeutic Treatment of SARS-Coronavirus Viral Load

Administration of an effective amount of a pharmaceutical composition comprising fenretinide (such as LAU-7b) is undertaken with a human patient experiencing a SARS-coronavirus infection; following which the subject subsequently exhibits reduced viral load.

Administration is undertaken by providing the patient an oral formulation comprising a pharmaceutically acceptable salt of fenretinide or analogs thereof such as LAU-7b; and it is contemplated to optionally include a pharmaceutically acceptable excipient as part of the oral formulation. Within a period of time, up to about 21 days, viral load is reduced in the patient.

### EXAMPLE 7

### Therapeutic Treatment of SARS-Coronavirus Inflammatory Response

Administration of an effective amount of a pharmaceutical composition comprising fenretinide (such as LAU-7b) is undertaken with a human patient experiencing a SARS-coronavirus infection; following which the subject subsequently exhibits an improved immunological response, namely reduced systemic, and/or pulmonary, inflammation.

Administration is undertaken by providing the patient an oral formulation comprising a pharmaceutically acceptable salt of fenretinide or analogs thereof such as LAU-7b; and it is contemplated to optionally include a pharmaceutically acceptable excipient as part of the oral formulation. Within a period of time, up to about 21 days, the patient exhibits improved immunological response, namely reduced systemic, and/or pulmonary, inflammation.

### EXAMPLE 8

### Prophylactic Treatment of SARS-Coronavirus Infection

Administration of an effective amount of a pharmaceutical composition comprising fenretinide (such as LAU-7b) is undertaken with a human patient prior to confirmation of SARS-coronavirus infection, following which the patient exhibits reduced symptoms of SARS-coronavirus infection or reduced severity of symptoms and/or disease complications associated with SARS-coronavirus infections, such as pneumonia, need for hospitalization, ARDS, need for mechanical ventilation, as compared to non-treated subjects.

Administration is undertaken by providing the patient an oral formulation comprising a pharmaceutically acceptable salt of fenretinide or analogs thereof such as LAU-7b; and it is contemplated to optionally include a pharmaceutically acceptable excipient as part of the oral formulation. Within a period of time, up to about 21 days, the patient exhibits reduced symptoms of SARS-coronavirus, pneumonia, ARDS and/or less or no hospitalization days required, and/or no mechanical ventilation required, as compared to non-treated subjects at a similar time point.

### EXAMPLE 9

### Prophylactic Treatment of SARS-Coronavirus Related Pneumonia

Administration of an effective amount of a pharmaceutical composition comprising fenretinide (such as LAU-7b) is undertaken with a human patient prior to onset of pneumonia associated with the SARS-coronavirus infection, following which the patient exhibits reduced or no symptoms of pneumonia compared to non-treated subjects.

Administration is undertaken by providing the patient an oral formulation comprising a pharmaceutically acceptable salt of fenretinide or analogs thereof such as LAU-7b; and it is contemplated to optionally include a pharmaceutically acceptable excipient as part of the oral formulation. Within a period of time, up to about 21 days, the patient exhibits reduced or no symptoms of pneumonia compared to non-treated subjects at a similar time point.

### EXAMPLE 10

### Prophylactic Treatment of SARS-Coronavirus Related ARDS

Administration of an effective amount of a pharmaceutical composition comprising fenretinide (such as LAU-7b) is undertaken with a human patient prior to onset of ARDS, following which the patient exhibits reduced or no symptoms of ARDS compared to non-treated subjects.

Administration is undertaken by providing the patient an oral formulation comprising a pharmaceutically acceptable salt of fenretinide or analogs thereof such as Lau-7b; and it is contemplated to optionally include a pharmaceutically acceptable excipient as part of the oral formulation. Within a period of time, up to about 21 days, the patient exhibits reduced or no symptoms of ARDS compared to non-treated subjects at a similar time point.

### EXAMPLE 11

### Therapeutic Treatment of ARDS.

Administration of an effective amount of a pharmaceutical composition comprising fenretinide (such as Lau-7b) is undertaken with a human patient following onset of ARDS, following which the patient exhibits reduced symptoms of ARDS compared to non-treated subjects.

Administration is undertaken by providing the patient an oral formulation comprising a pharmaceutically acceptable salt of fenretinide or analogs thereof such as Lau-7b; and it is contemplated to optionally include a pharmaceutically acceptable excipient as part of the oral formulation. Within a period of time, up to about 21 days, the patient exhibits reduced symptoms of ARDS compared to non-treated subjects at a similar time point.

While particular embodiments of the present invention have been described in the foregoing, it is to be understood that other embodiments are possible within the scope of the invention and are intended to be included herein. It will be clear to any person skilled in the art that modifications of and adjustments to this invention, not shown, are possible without departing from the spirit of the invention as demonstrated through the exemplary embodiments. The invention is therefore to be considered limited solely by the scope of the appended claims.

1-138. (canceled)

139. A method of treating a SARS-coronavirus infection in a human comprising administration to said human of a therapeutically effective amount of fenretinide, fenretinide analog or pharmaceutically acceptable salt thereof

140. The method of claim 139 wherein the therapeutically effective amount of fenretinide, fenretinide analog or pharmaceutically acceptable salt thereof comprises 10 mg to 300 mg of fenretinide.

141. The method of claim 140, wherein the fenretinide is provided as LAU-7b.

142. The method of claim 141 wherein the administration comprises oral administration to said human of a dose of LAU-7b comprising 300 mg of fenretinide, once per day for 3 days, followed by oral administration to said human of a dose of LAU-7b comprising 200 mg of fenretinide, once per day, for 11 days.

143. A method of treating a SARS-coronavirus associated pneumonia in a human comprising administration to said human of a therapeutically effective amount of fenretinide, fenretinide analog or pharmaceutically acceptable salt thereof

144. The method of claim 143 wherein the therapeutically effective amount of fenretinide, fenretinide analog or pharmaceutically acceptable salt thereof comprises 10 mg to 300 mg of fenretinide.

145. The method of claim 144, wherein the fenretinide is provided as LAU-7b.

146. The method of claim 145 wherein the administration comprises oral administration to said human of a dose of LAU-7b comprising 300 mg of fenretinide, once per day for 3 days, followed by oral administration to said human of a dose of LAU-7b comprising 200 mg of fenretinide, once per day, for 11 days.

147. A method of prophylaxis of SARS-coronavirus infection in a human comprising administration to said human of a therapeutically effective amount of fenretinide, fenretinide analog or pharmaceutically acceptable salt thereof.

148. The method of claim 147 wherein the therapeutically effective amount of fenretinide, fenretinide analog or pharmaceutically acceptable salt thereof comprises 10 mg to 300 mg of fenretinide.

149. The method of claim 148, wherein the fenretinide is provided as LAU-7b.

150. A method of prophylaxis of acute respiratory distress syndrome in a human comprising administration to said human of a therapeutically effective amount of fenretinide, fenretinide analog or pharmaceutically acceptable salt thereof

151. The method of claim 150 wherein the therapeutically effective amount of fenretinide, fenretinide analog or pharmaceutically acceptable salt thereof comprises 10 mg to 300 mg of fenretinide.

152. The method of claim 151, wherein the fenretinide is provided as LAU-7b.

153. The method of claim 152 wherein the administration comprises oral administration to said human of a dose of LAU-7b comprising 300 mg of fenretinide, once per day for 3 days, followed by oral administration to said human of a dose of LAU-7b comprising 200 mg of fenretinide, once per day, for 11 days.

154. The method of claim 151 wherein the acute respiratory distress syndrome is associated with SARS-coronavirus.

155. A method of preventing overactive lung inflammatory response in a human comprising administration to said human of a therapeutically effective amount of fenretinide, fenretinide analog or pharmaceutically acceptable salt thereof

156. The method of claim 155 wherein the therapeutically effective amount of fenretinide, fenretinide analog or pharmaceutically acceptable salt thereof comprises 10 mg to 300 mg of fenretinide.

157. The method of claim 156, wherein the fenretinide is provided as LAU-7b.

158. The method of claim 157 wherein the administration comprises oral administration to said human of a dose of LAU-7b comprising 300 mg of fenretinide, once per day for 3 days, followed by oral administration to said human of a dose of LAU-7b comprising 200 mg of fenretinide, once per day, for 11 days.

159. The method of claim 158 wherein the overactive lung inflammatory response is associated with SARS-coronavirus.

160. A method of reducing SARS-coronavirus viral load in a human comprising administration to said human of a therapeutically effective amount of fenretinide, fenretinide analog or pharmaceutically acceptable salt thereof in combination with administration to said human of a therapeutically effective amount of a delayed chain terminator antiviral compound.

161. The method of claim 160 wherein the delayed chain terminator antiviral compound is selected from the group comprising remdesivir, penciclovir, cidofovir and entecavir.

162. The method of claim 161 wherein the therapeutically effective amount of fenretinide, fenretinide analog or pharmaceutically acceptable salt thereof comprises 10 mg to 300 mg of fenretinide.

163. The method of claim 162, wherein the fenretinide is provided as LAU-7b.

164. A method of treating hypoxemia in a human comprising administration to said human of a therapeutically effective amount of fenretinide, fenretinide analog or pharmaceutically acceptable salt thereof

165. The method of claim 164 wherein the therapeutically effective amount of fenretinide, fenretinide analog or pharmaceutically acceptable salt thereof comprises 10 mg to 300 mg of fenretinide.

166. The method of claim 165, wherein the fenretinide is provided as LAU-7b.

167. The method of claim 166 wherein the administration comprises oral administration to said human of a dose of LAU-7b comprising 300 mg of fenretinide, once per day for 3 days, followed by oral administration to said human of a dose of LAU-7b comprising 200 mg of fenretinide, once per day, for 11 days.

168. A method of prophylaxis of hypoxemia in a human comprising administration to said human of a therapeutically effective amount of fenretinide, fenretinide analog or pharmaceutically acceptable salt thereof.

169. The method of claim 168 wherein the therapeutically effective amount of fenretinide, fenretinide analog or pharmaceutically acceptable salt thereof comprises 10 mg to 300 mg of fenretinide.

170. The method of claim 169, wherein the fenretinide is provided as LAU-7b.

171. The method of claim 170 wherein the administration comprises oral administration to said human of a dose of LAU-7b comprising 300 mg of fenretinide, once per day for 3 days, followed by oral administration to said human of a dose of LAU-7b comprising 200 mg of fenretinide, once per day, for 11 days.

172. A method of prophylaxis of SARS-coronavirus associated pneumonia in a human comprising administration to said human of a therapeutically effective amount of fenretinide, fenretinide analog or pharmaceutically acceptable salt thereof.

173. The method of claim 172 wherein the therapeutically effective amount of fenretinide, fenretinide analog or pharmaceutically acceptable salt thereof comprises 10 mg to 300 mg of fenretinide.

174. The method of claim 173, wherein the fenretinide is provided as LAU-7b.

175. A method of treating a condition associated with reticulocytopenia in a human comprising administration to said human of a therapeutically effective amount of fenretinide, fenretinide analog or pharmaceutically acceptable salt thereof.

176. The method of claim 175 wherein the therapeutically effective amount of fenretinide, fenretinide analog or pharmaceutically acceptable salt thereof comprises 10 mg to 300 mg of fenretinide.

177. The method of claim 176, wherein the fenretinide is provided as LAU-7b.

178. The method of claim 177 wherein the administration comprises oral administration to said human of a dose of LAU-7b comprising 300 mg of fenretinide, once per day for 3 days, followed by oral administration to said human of a dose of LAU-7b comprising 200 mg of fenretinide, once per day, for 11 days.
